(19) United States
(12) Patent Application Publication
(10) Pub. No.: US 2013/0244963 A1
(43) Pub. Date: Sep. 19, 2013

(54) COMBINATION THERAPY WITH 4-(3-(2H-1,2,3-TRIAZOL-2-YL)PHENYLAMINO)-2-((1R,2S)-2-AMINOCYCLOHEXYLAMINO)PYRIMIDINE-5-CARBOXAMIDE

(71) Applicants: Uma Sinha, San Francisco, CA (US); Anjali Pandey, Fremont, CA (US)

(72) Inventors: Uma Sinha, San Francisco, CA (US); Anjali Pandey, Fremont, CA (US)

(73) Assignee: Portola Pharmaceuticals, Inc., South San Francisco, CA (US)

(21) Appl. No.: 13/823,699

(22) PCT Filed: Sep. 30, 2011
(86) PCT No.: PCT/US2011/054233

§ 371 Date: May 28, 2013

Related U.S. Application Data
(60) Provisional application No. 61/388,568, filed on Sep. 30, 2010.

(51) Int. Cl. A61K 31/506; A61K 45/06; A61K 31/704; A61K 31/519; A61K 31/573

(52) U.S. Cl. CPC A61K 31/506; A61K 31/519; A61K 31/573; A61K 31/704; A61K 45/06
USPC 514/34; 514/275; 514/249; 514/171

(57) ABSTRACT

The present invention is directed to pharmaceutical compositions and methods of using combination therapies containing a SYK inhibitor, or a pharmaceutically acceptable salt thereof, and a antineoplastic or antiinflammatory agent for the treatment of inflammatory, autoimmune and cell proliferative diseases, such as allergic reaction, transplant rejection, rheumatoid arthritis (RA), lupus, multiple sclerosis (MS) or psoriasis undesired acute myeloid leukemia (AML), chronic lymphocytic leukemia (CLL), non-Hodgkin lymphoma (NHL) (including diffuse large B cell lymphoma (DLBCL)), mantle cell lymphoma, acute lymphocytic leukemia (ALL), follicular lymphoma, Burkitt's lymphoma, small Lymphocytic (SLL), Lymphoma, multiple myeloma, asthma, vasculitis, Idiopathic thrombocytopenic purpura (ITP), Heparin Induced Thrombocytopenia (HIT) and hemolytic anemia.

### CROSS-REFERENCES TO RELATED APPLICATIONS

This application claims the benefit of priority under 35 U.S.C. 119(e) to U.S. Provisional Application No. 61/388,568 filed on Sep. 30, 2010 which are herein incorporated in their entirety by reference.

### BACKGROUND OF THE INVENTION

The present invention relates generally to novel compositions and methods of using a combination of 4-(3-(2H-1,2,3-triazol-2-yl)phenylamino)-2-(1R,2S)-2-aminocyclohexylamino)pyrimidine-5-carboxamide (Compound 1, P505-15, PRT 062607, 2607). The present invention also relates to novel compositions and methods using a combination of Compound 1 with an antineoplastic or anti-inflammatory agent for the treatment of inflammatory, autoimmune and cell proliferative diseases. The invention is also directed to methods of making the compositions described herein.

### STATE OF THE ART

Cell-proliferative diseases are a major cause of death in the industrialized world. Examples include acute myeloid leukemia (AML), chronic lymphocytic leukemia (CLL), non-Hodgkin lymphoma (NHL) (including diffuse large B cell lymphoma (DLBCL)), mantle cell lymphoma, acute lymphocytic leukemia (ALL), follicular lymphoma, Burkitt's lymphoma, small Lymphocytic Lymphoma (SLL) of which CLL is the most common form of adult leukemia. Single compounds well as selective combinations of purine and pyrimidine analogs are known to increase remission rates, especially in patients with relapsed leukemias. Antineoplastic agents and antiinflammatories are often used in, mainly pairwise, combination.

Syk is important for the activation of B-cells via a B-cell antigen receptor and is involved in the phosphatidylinositol metabolism and increase in the intracellular calcium concentration caused by the antigen receptor stimulation (Hutchcroft, J E. et al., J. Biol. Chem., 267:8613-8619, 1992; and Takata, M. et al., EMBO J., 13:1341-1349, 1994). Thus, syk inhibitors may be used to control the function of B-cells and are, therefore, expected to serve as therapeutic agents for antibody-related diseases.

In addition, JAK/STAT signaling has been implicated in the mediation of many abnormal immune responses such as allergies, asthma, autoimmune diseases such as transplant (allograft) rejection, rheumatoid arthritis, multiple sclerosis, as well as in solid and hematologic malignancies such as leukemia and lymphomas. For a review of the pharmaceutical intervention of the JAKISTAT pathway see Frank, (1999), Mol. Med. 5:432:456 and Seidel et al., (2000), Oncogene 19:2645-2656.

Recent comparative genomic hybridization studies have identified syk as another gene important in the pathogenesis of Mantle Cell Lymphoma (MCL) (Chen, R. et al. Journal of Clinical Oncology, 2007 ASCO Annual Meeting Proceedings (Post-Meeting Edition). Vol 25, No 18S (June 20 Supplement), 2007: 8056). MCL represents 5-10% of all non-Hodgkin lymphomas and it is a difficult form of lymphoma to treat. It has the worst prognosis among the B cell lymphomas with median survival of three years. It has been reported that Syk is overexpressed in MCL (Rinaldi, A, et. al, Br. J. Haematol., 2006; 132:303-316) and that Syk mediates mTOR (mammalian target of Rapamycin) survival signals in follicular, mantle cell, Burkitt's, and diffuse large B-cell non-Hodgkin lymphomas (Leseux, L., et. al, Blood, 2006; 108:4156-4162).

Several lines of evidence suggest that many B-cell lymphomas depend upon B-cell receptor (BCR)-mediated survival signals. BCR signaling induces receptor oligomerization and phosphorylation of Igα and β immunoreceptor tyrosine-based activated motifs by SRC family kinases. ITAM phosphorylation results in the recruitment and activation of syk that initiates downstream events and amplifies the original BCR signal. Given the role of tonic BCR signaling in normal B cell and syk-dependent survival of non-Hodgkin lymphoma cell lines in vitro (Chen, L., et. al, Blood, 2006; 108:3428-3433), syk inhibition is a promising rational treatment target for certain B-cell lymphomas and chronic lymphocytic leukemia (CLL) (Stefania Gobessi, Luca Laurenti, Pablo Longo, Laura Carsetti, Giuseppe Leone, Dimitar G. Efremov, Constitutive activation of the protein tyrosine kinase Syk in Chronic Lymphocytic Leukemia B-cells, Blood, 2007, 110, Abstract 1123). Recent data shows that administration of a multikinase inhibitor which inhibits syk, may have significant clinical activity in CLL patients (Friedberg et al, Blood 2008; 112(11); Blood 2010; 115:2578-2585; Hahn et al., Blood, 2007, 110, Abstract 209).

The oncogenic potential of the spleen tyrosine kinase (Syk) has been described in a number of different settings. Clinically, Syk over-expression is reported in Mantle Cell Lymphoma (Rinaldi, A, et. al, Br. J. Haematol., 2006; 132:303-316) and the TEL-Syk fusion protein (Translocated ETS Leukemia) generated by a chromosomal translocation (t(9;12)(q22;p12)) leads to increased Syk activity and is associated with myelodysplastic syndrome (Kuno, Y., et. al, Blood, 2001; 97:1050-1055). Leukemia is induced in mice by adoptively transferring bone marrow cells that express human TEL-Syk (Wossning, T., JEM, 2006; 203:2829-2840). Further, in mouse primary bone marrow cells, over-expression of Syk results in IL-7 independent growth in culture (Wossning, T., et. al, JEM, 2006; 203:2829-2840).

Interestingly, Syk signaling appears to be required for B-cell development and survival in humans and mouse. Inducible loss of the B-cell receptor (Lam, K., et. al, Cell, 1997; 90:1073-1083) or Igα (Kraus, M., et. al, Cell, 2004; 117:787-800) results in loss of peripheral B-cells in mice. Over-expression of the protein tyrosine phosphatase PTP-RO, which is known to negatively regulate Syk activity, inhibits proliferation and induces apoptosis in cell lines derived from non-Hodgkin lymphomas (Chen, L., et. al, Blood, 2006; 108:3428-3433). Finally, B-cell lymphomas rarely exhibit loss of BCR expression, and anti-idiotype therapy rarely leads to resistance (Kuppers, R. Nat Rev Cancer, 2005; 5:251-262).

Engagement of the antigen-specific B cell receptor (BCR) activates multiple signaling pathways that ultimately regulate the cells activation status, promoting survival and clonal expansion. Signaling through the BCR is made possible by its association with two other members of the immunoglobulin super-family; Igα and Igβ, each bearing an immuno-tyrosine based activation motif (ITAM) (Jumaa, Hendriks et al. Annu Rev Immunol 23: 415-45 (2005). The ITAM domain is directly phosphorylated by Src family kinases in response to BCR engagement. The spleen tyrosine kinase (Syk) docks with and phosphorylates the ITAM, a process that enhances its kinase activity, resulting in Syk autophosphorylation and tyrosine phosphorylation of multiple downstream substrates (Rolli, Gallwitz et al. Mol Cell 10(5): 1057-69 (2002). This signaling pathway is active in B cells beginning at the transition from pro- to pre-B cell stage of development, when the newly formed pre-BCR is expressed. In fact, B cell development arrests at the pro-B cell stage in Syk knockout mice (Cheng, Rowley et al. 1995; Turner, Mee et al. Nature 378(6554): 303-6 (1995). Inducible loss of the B cell receptor (Lam, Kuhn et al. Cell 90(6): 1073-83 (1997) or Igα (Kraus, Alimzhanov et al. Cell 117(6): 787-800 (2004) results in loss of peripheral B cells in mice. Human B cells also appear to require Syk for proliferation and survival. Over-expression of the protein tyrosine phosphatase PTP-RO, a negative regulator of Syk activity, inhibits proliferation and induces apoptosis in cell lines derived from NHL (Chen, Juszczynski et al. Blood 108(10): 3428-33 (2006). Knock down of Syk by siRNA in the NHL line SUDHL-4 led to a block in the G1/S transition of the cell cycle (Gururajan, Dasu et al. J Immunol 178(1): 111-21 (2007). Together, these data suggest that Syk signaling is required for the development, proliferation, and even survival of human and mouse B cells.

Conversely, the oncogenic potential of Syk has been described in a number of different settings. Clinically, Syk over-expression is reported in Mantle Cell Lymphoma (Rinaldi, Kwee et al. Br J Haematol 132(3): 303-16 (2006) and the TEL-Syk fusion protein (Translocated ETS Leukemia) generated by a chromosomal translocation (t(9;12)(q22;p12)) leads to increased Syk activity and is associated with myelodysplastic syndrome (Kuno, Abe et al. Blood 97(4): 1050-5 (2001). Leukemia is induced in mice by the adoptive transfer of bone marrow cells that express human TEL-Syk (Wossning, Herzog et al. J. Exp. Med. 203(13): 2829-40 (2006). Further, in mouse primary bone marrow cells, over-expression of Syk results in IL-7 independent growth in culture (Wossning, Herzog et al. 2006).

Consistently, Syk was reported to mediate mTOR (mammalian target of Rapamycin) survival signals in follicular, mantle cell, Burkitt's, and diffuse large B-cell NHL (Leseux, Hamdi et al. Blood 108(13): 4156-62 (2006). Additional recent studies also suggest that Syk-dependant survival signals may play a role in B-cell malignancies, including DLBCL, mantle cell lymphoma and follicular lymphoma (Gururajan, Jennings et al. 2006; Irish, Czerwinski et al. J. Immunol. 176(10): 5715-9 (2006). Given the role of tonic BCR signaling in normal B cells and Syk-dependent survival of NHL cell lines in vitro, the specific inhibition of Syk may prove promising for the treatment of certain B-cell lymphomas.

B-cell receptor (BCR) associated kinases have recently been shown to play a role in the pathogenesis of B cell malignancies. Spleen tyrosine kinase (SYK) is of particular interest as its activation results in enhanced proliferation and survival of B-cells. Analysis of NHL cell lines and primary CLL samples have shown that SYK is persistently phosphorylated and that SYK inhibition results in abrogation of downstream kinase activity, leading to apoptosis. The kinase inhibitor Fostamatinib disodium (FosD, R788/R406), which has shown clinical activity in heavily pre-treated NHL and CLL patients, exhibits inhibitory activity against SYK (IC₅₀=40 nM) but also inhibits a broad spectrum of other kinase targets.

U.S. Patent Publication No. 2010/0048567, titled “INHIBITORS OF SYK PROTEIN KINASE,” filed Apr. 16, 2009, the contents of which are incorporated herein by reference in its entirety, discloses a novel small-molecule SYK inhibitor compound, 4-(3-(2H-1,2,3-triazol-2-yl)phenylamino)-2-(1R,2S)-2-aminocyclohexylamino)pyrimidine-5-carboxamide, (Compound 1), which has the following structure:

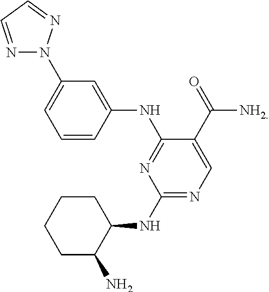 [SMILES: NC(=O)c1cnc(N[C@@H]2CCCC[C@@H]2N)nc1Nc1cccc(-n2nccn2)c1]

Compound 1 acts as a potent and selective inhibitor of SYK.

Recently, fostamatinib (R788/R406) (Rigel Pharmaceuticals) was reported to inhibit ITAM signaling in response to various stimuli, including FcεR1 and BCR induced Syk activation (Braselmann, Taylor et al. J. Pharmacol. Exp. Ther. 319(3): 998-1008 (2006). Interestingly, this ATP-competitive inhibitor of Syk was also active against Flt3, cKit, and JAK kinases, but not against Src kinsase (Braselmann, Taylor et al. 2006). Activating mutations to Flt3 are associated with AML and inhibition of this kinase is currently under clinical development (Burnett and Knapper Hematology Am. Soc. Hematol. Educ. Program 2007: 429-34 (2007); Golub and Stegmaier et al.). Over-activation of the tyrosine kinase cKit is also associated with hematologic malignancies, and a target for cancer therapy (Heinrich, Griffith et al. Blood 96(3): 925-32 (2000). Similarly, JAK3 signaling is implicated in leukemias and lymphomas, and is currently exploited as a potential therapeutic target (Heinrich, Griffith et al. 2000). Importantly, the multi-kinase inhibitory activity of R406 attenuates BCR signaling in lymphoma cell lines and primary human lymphoma samples, resulting in apoptosis of the former (Chen, Monti et al. Blood 111(4): 2230-7 (2008). Further, a phase II clinical trial reported favorable results by this compound in refractory NHL and chronic lymphocytic leukemia (Friedberg et al, Blood 2008; 112(11)). Although the precise mechanism of action is unclear for R406, the data suggest that inhibition of kinases that mediate survival signaling in lymphocytes is clinically beneficial.

Additional recent studies also suggest that Syk-dependent survival signals may play a role in B-cell malignancies, including DLBCL, mantle cell lymphoma and follicular lymphoma (see e.g., S. Linfengshen et al. Blood, February 2008; 111: 2230-2237; J. M. Irish et al. Blood, 2006; 108: 3135-3142; A. Renaldi et al. Brit J. Haematology, 2006; 132: 303-316; M. Guruoajan et al. J. Immunol, 2006; 176: 5715-5719; L. Laseux et al. Blood, 2006; 108: 4156-4162.

In view of the relatively high toxicities associated with the treatment of proliferative diseases, especially leukemias, by chemotherapeutics such as those mentioned above, it remains a goal to devise novel treatment schedules or novel combinations that in principle allow for treatment with lower doses of the individual compounds, thus making it possible to allow for diminuation of the toxicities individually associated with highly toxic compounds. In addition, there remains a need in the art for methods for treating conditions in a patient, such as CLL that is currently incurable. Furthermore, specific proliferative diseases and/or specific patient groups (e.g. related to sex or especially age, such as in case of pediatric or geriatric use, or patients where the proliferating cells became refractory to treatment with known chemotherapeutics or combinations thereof) may require more specific, even individual therapeutic regimens.

There is also a need for combination of two different drugs that act by different mechanisms (e.g., a Syk inhibitor (Compound 1) and a antineoplastic or antiinflammatory agent optionally in combination with another anti-inflammatory agent, an immunosuppressant agent, an antimetabolic agent a hormone; an antimicrobial agent; another antineoplastic or anti-inflammatory agent or agents, as such a triple combination (cyclophosphamide, mitoxantrone, dexamethasone, rituximab, cytarabine, granulocyte colony-stimulating factor, Trimethoprim/sulfamethoxazole, co-trimoxazole, and pentamidine) as presently used as separate entities during chemotherapy to increase efficacy and/or improve safety more than any of these drugs used alone or a combination of any two of these agents. The present invention satisfies this and other needs.

### BRIEF SUMMARY OF THE INVENTION

This invention provides methods and pharmaceutical compositions of combined therapies comprising a SYK inhibitor, having the structure:

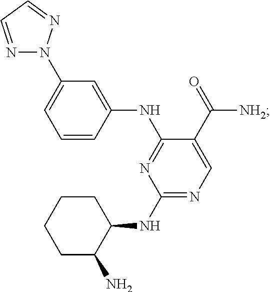 [SMILES: NC(=O)c1cnc(N[C@@H]2CCCC[C@@H]2N)nc1Nc1cccc(-n2nccn2)c1]

which has the chemical name 4-(3-(2H-1,2,3-triazol-2-yl)phenylamino)-2-((1R,2S)-2-aminocyclohexylamino)pyrimidine-5-carboxamide, and is referred to throughout as “Compound 1”.

It is contemplated based on experimental results that a combination of Compound 1 with an antineoplastic or antiinflammatory agent will produce improved antineoplastic effect over any of the agents alone.

Accordingly, the present invention provides a method for treating inflammatory, autoimmune and cell proliferative diseases selected from the group consisting of allergic reaction, transplant rejection, rheumatoid arthritis (RA), lupus, multiple sclerosis (MS), psoriasis, leukemia, a lymphoma, myeloproliferative disorders, hematological malignancies, and chronic idiopathic myelofibrosis comprising administering to a mammal a therapeutically effective amount of an agent 4-(3-(2H-1,2,3-triazol-2-yl)phenylamino)-2-((1R,2S)-2-aminocyclohexylamino)pyrimidine-5-carboxamide, or a pharmaceutically acceptable salt thereof; and an antineoplastic or anti-inflammatory agent.

In one aspect, the present invention provides a method, wherein the antineoplastic or antiinflammatory agent is selected from the group consisting of a topoisomerase II inhibitor, aribonucleotide reductase inhibitor, a DNA polymerase inhibitor, and combinations thereof.

In another aspect, the present invention provides a method, wherein the antineoplastic or antiinflammatory agent is selected from the group consisting of is selected from the group consisting of methotrexate, dexamethazone, prednisone, and the like.

In another aspect, the present invention provides a method for treating NHL, wherein the antineoplastic or antiinflammatory agent is selected from the group consisting of cyclophosphamide/cytoxan, adriamycin/doxorubicin/hydroxorubicin/danorubicin, vincristine and prednisone (CHOP).

In another aspect, the present invention provides a method for treating NHL, wherein the antineoplastic or antiinflammatory agent is selected from the group consisting of cyclophosphamide/cytoxan, adriamycin/doxorubicin/hydroxorubicin/danorubicin, vincristine and prednisone (CHOP) and rituximab.

In another aspect, the present invention provides a method for treating NHL, wherein the antineoplastic or antiinflammatory agent is selected from the group consisting of bortezomib and HDAC inhibitors.

In another aspect, the present invention provides a method for treating NHL, wherein the antineoplastic or antiinflammatory agent is selected from the group consisting of methotrexate, bleomycin, adriamycin, cyclophosphamide, vincristine and dexamathasone (m-BACOD). In one aspect, the antineoplastic or antiinflammatory agent is methotrexate.

In another aspect, the present invention provides a method for treating NHL or acute T-cell leukemia wherein the antineoplastic or antiinflammatory agent is selected from the group consisting of etoposide, prednisone, vincristine, cyclophosphamide and doxorubicin (EPOCH).

In another aspect, the present invention provides a method for treating NHL, wherein the antineoplastic or antiinflammatory agent is selected from the group consisting of ifosfamide, carboplatin and etoposide (ICE).

In another aspect, the present invention provides a method for treating NHL, wherein the antineoplastic or antiinflammatory agent is selected from the group consisting of dexamethasone, high dose cytarabine andcisplatin (DHAP).

In another aspect, the present invention provides a method for treating NHL, wherein the antineoplastic or antiinflammatory agent is Mitoxantrone.

In another aspect, the present invention provides a method for treating NHL, wherein the antineoplastic or antiinflammatory agent is Flavopiridol.

In another aspect, the present invention provides a method for treating NHL or relapsed or refractory peripheral T-cell lymphoma, wherein the antineoplastic or antiinflammatory agent is Pralatrexate (Folotyn).

In another aspect, the present invention provides a method for treating CLL, wherein the antineoplastic or antiinflammatory agent is selected from the group consisting of Cyclophosphamide, chlorambucil, bendamustine and gemcitabine.

In another aspect, the present invention provides a method for treating CLL, wherein the antineoplastic or antiinflammatory agent is selected from the group consisting of Revlimid and thalidomide.

In another aspect, the present invention provides a method for treating CLL, wherein the antineoplastic or antiinflammatory agent is Rituximab.

In another aspect, the present invention provides a method for treating CLL, wherein the antineoplastic or antiinflammatory agent is selected from the group consisting of Pentostatin and cladribine.

In another aspect, the present invention provides a method for treating CLL, wherein the antineoplastic or antiinflammatory agent is Allopurinol.

In another aspect, the present invention provides a method for treating CLL, wherein the antineoplastic or antiinflammatory agent is Alemtuzumab (Campath-1H).

In another aspect, the present invention provides a method for treating CLL, wherein the antineoplastic or antiinflammatory agent is selected from the group consisting of Oxaliplatin cytarabine and rituximab (OFAR).

In another aspect, the present invention provides a method for treating CLL, wherein the antineoplastic or antiinflammatory agent is Flavopiridol.

In another aspect, the present invention provides a method for treating CLL, wherein the antineoplastic or antiinflammatory agent is mitoxantrone.

In another aspect, the present invention provides a method for treating CLL, wherein the antineoplastic or antiinflammatory agent is Ofatumumab (HuMax CD20).

In another aspect, the present invention provides a method for treating CLL, wherein the antineoplastic or antiinflammatory agent is Lenalidomide

In another aspect, the present invention provides a method for treating CLL, T-cell lymphoblastic lymphoma, acute lymphoblastic leukemia, or NHL, wherein the antineoplastic or antiinflammatory agent is Nelarabine.

In another aspect, the present invention provides a method for treating CLL, wherein the antineoplastic or antiinflammatory agent is Navelbine.

In another aspect, the present invention provides a method wherein an additional therapeutic agent is used. In one aspect the additional therapeutical agent is an immune modulator, an antimalarial, an anti-hyperuricemia agent, a blood cell producing agent, an antibiotic and the like.

Examples of immune modulators include, but not limited to tumor necrosis factor α (TNFα) inhibitors, such as infliximab, adalimumab, anakinra, etanercept and the like; anti-IL6 agents, such as actemra (tocilimuzab) and CNTO 328; abatacept, certolizumab pegol, mycophenolate mofetil; azathioprene; B lymphocyte stimulator (BLyS) inhibitors such as belimumab; thrombopoietin receptor agonists such as romiplostim, and the like; eltrombopag; Bruton's tyrosine kinase (BTK) inhibitors, such as PCI-32765 and the like; protein kinase C (PKC) inhibitors such as dexamethasone and the like; prednisone; belatacept; steroids; non-steroidal anti-inflammatory drugs (NSAIDS), such as indomethacin; anti-psoriasis agents such as vitamin D analogs such as calcipotriene, and the like; antihistamines, such as clemastine, diphenhydramine, doxylamine, loratadine, desloratadine, fexofenadine, pheniramine, cetirizine, ebastine, promethazine, chlorpheniramine. levocetirizine, olopatadine, quetiapine, meclizine, dimenhydrinate, embramine, dimethindene, dexchlorpheniramine, cimetidine famotidine, ranitidine, nizatidine, roxatidine, lafutidine and the like; anti-asthma agent such as leukotriene receptor antagonists including but not limited to montelukast; anti Rhesus (Rh) factor D immunoglobulins; calcineurin inhibitors such as voclosporin; anti-multiple sclerosis agents such as copaxone, beta interferon beta-1a, mitoxantrone, natalizumab, tacrolimus, and the like; and JAK kinase inhibitors such as tofacitinib (CP-690550), tasocitinib, VX-509, Ruxolitinib (INCB18424), fostamatinib (R788), and the like.

Examples of antimalarials, include hydroxychloroquine, and the like.

Examples of anti-hyperuricemia agents include colchicine, allopurinol, probenecid, and the like.

Examples of blood cell producing agents include granulocyte colony stimulating factor, Erythropoietin, and the like.

Examples of general antibiotic include co-trimoxazole, pentamidine, and the like.

In still another aspect, the present invention provides a method, wherein the method is used in combination with radiation therapy.

In still another aspect, the present invention provides a method, wherein the subjects of the method have previously had autologus stem cell transplantation.

In still another aspect, the present invention provides a method, wherein the antineoplastic or antiinflammatory agent is selected from Idarubicine and ara-C, or a pharmaceutically acceptable salt thereof.

In still another aspect, the present invention provides a method, wherein the antineoplastic or antiinflammatory agent is not fludarabine, or a pharmaceutically acceptable salt thereof.

The present invention also provides a method, wherein the pharmaceutically acceptable salt of 4-(3-(2H-1,2,3-triazol-2-yl)phenylamino)-2-((1R,2S)-2-aminocyclohexylamino)pyrimidine-5-carboxamide is the hydrochloride or acetate salt.

In another aspect, the present invention provides a method, further comprising administering a therapeutic agent selected from the group consisting of: an anti-inflammatory agent, an immunosuppressant agent, an antimetabolic agent a hormone; an antimicrobial agent; another antineoplastic or antiinflammatory agent and combinations thereof.

In another aspect, the present invention provides a method, wherein the therapeutic agent is selected from the group consisting of: cyclophosphamide, mitoxantrone, dexamethasone, rituximab, cytarabine, granulocyte colony-stimulating factor; co-trimoxazole, and pentamidine.

In another aspect, the present invention provides a method, wherein at least one of the agents is administered in a sub-therapeutic dosage.

In another aspect, the present invention provides a method, wherein at least two of the agents are administered in sub-therapeutic dosages.

In another aspect, the present invention provides a method, wherein all of the agents are administered in sub-therapeutic dosages.

In another aspect, the present invention provides a method, wherein at least two of the agents are administered simultaneously.

In another aspect, the present invention provides a method, wherein at least two of the agents are administered separately.

In another aspect, the present invention provides a method, wherein at least two of the agents are administered sequentially.

In still another aspect, the present invention provides a method, wherein said cell proliferative disorder is acute myeloid leukemia (AML), chronic lymphocytic leukemia (CLL), non-Hodgkin lymphoma (NHL), diffuse large B cell lymphoma, mantle cell lymphoma, follicular lymphoma, Burkitt's lymphoma, small Lymphocytic Lymphoma (SLL).

In another aspect, the present invention provides a method, wherein said inflammatory or autoimmune disease is an allergic reaction, transplant rejection, rheumatoid arthritis (RA), lupus, multiple sclerosis (MS) or psoriasis.

The present invention also provides a composition for treating inflammatory, autoimmune and cell proliferative diseases comprising administering to said mammal a therapeutically effective amount of an agent 4-(3-(2H-1,2,3-triazol-2-yl)phenylamino)-2-((1R,2S)-2-aminocyclohexylamino)pyrimidine-5-carboxamide, or a pharmaceutically acceptable salt thereof; and an antineoplastic or anti-inflammatory agent.

In another aspect, the present invention provides a composition, wherein the antineoplastic or anti-inflammatory agent is selected from the group consisting of a topoisomerase II inhibitor, aribonucleotide reductase inhibitor, a DNA polymerase inhibitor, and combinations thereof.

In one aspect, the present invention provides a method, wherein the antineoplastic or antiinflammatory agent is selected from the group consisting of is selected from the group consisting of methotrexate, dexamethazone, prednisone, cyclophosphamide/cytoxan, adriamycin/doxorubicin/hydroxorubicin/danorubicin, proteosome inhibitors such as bortezomib, HDAC inhibitors, bleomycin, vincristine, doxorubicin, ifosfamide, carboplatin, etoposide, cytarabine, cisplatin, Mitoxantrone, Pralatrexate (Folotyn), chlorambucil, bendamustine, gemcitabine, Revlimid, thalidomide and analogs, Pentostatin, cladribine, Allopurinol, Alemtuzumab (Campath-1H), Oxaliplatin, cytarabine, rituximab, Flavopiridol, mitoxantrone, anti-CD20 antibodies such as ofatumumab (HuMax CD20), Lenalidomide, Nelarabine and Navelbine; and the like.

In another aspect, the present invention provides a method, wherein the antineoplastic or antiinflammatory agent is selected from the group consisting of is selected from the group consisting of azacitidine; cladribine; decitabine; mercaptopurine; thioguanine; clofarabine; troxacitabine; and pentostatin; sulfosalazine; MLN9708, and the like; histone deacetylase (HDAC) inhibitors such as entinostat, and the like; bendamustine; chlorambucil; PI3k kinase inhibitors such as CAL-101, CAL-263, CAL-120, and the like; aurora kinase inhibitors such as MLN8237, PHA739358, and the like; anti CD52 antibodies, such as alemtuzumab, and the like; JAK inhibitors, such as tofacitinib (CP-690550), tasocitinib, VX-509, Ruxolitinib (INCB18424), fostamatinib (R788) and the like.

In another aspect, the present invention provides a composition, wherein the antineoplastic or antiinflammatory agent is selected from Idarubicine and ara-C, or a pharmaceutically acceptable salt thereof.

In another aspect, the present invention provides a composition, wherein the antineoplastic or antiinflammatory agent is not fludarabine, or a pharmaceutically acceptable salt thereof.

In another aspect, the present invention provides a composition, wherein the pharmaceutically acceptable salt of 4-(3-(2H-1,2,3-triazol-2-yl)phenylamino)-2-(1R,2S)-2-aminocyclohexylamino)pyrimidine-5-carboxamide is the hydrochloride or acetate salt.

In another aspect, the present invention provides a composition, further comprising administering a therapeutic agent selected from the group consisting of: an anti-inflammatory agent, an immunosuppressant agent, an antimetabolic agent a hormone; an antimicrobial agent; another antineoplastic or antiinflammatory agent and combinations thereof.

In another aspect, the present invention provides a composition, wherein the therapeutic agent is selected from the group consisting of: cyclophosphamide, mitoxantrone, dexamethasone, rituximab, cytarabine, granulocyte colony-stimulating factor; co-trimoxazole, and pentamidine.

In another aspect, the present invention provides a composition, wherein at least one of the agents is in a sub-therapeutic dosage.

In another aspect, the present invention provides a composition, wherein at least two of the agents are in sub-therapeutic dosages.

In another aspect, the present invention provides a composition, wherein all of the agents are in sub-therapeutic dosages.

In another aspect, the present invention provides a composition, wherein the composition is administered intravenously (e.g. injected), subcutaneously, or orally.

In another aspect, the present invention provides a composition found in the Examples.

In another aspect, the present invention provides a composition found in the Tables.

In another aspect, the present invention provides a composition found in the Figures.

In another aspect, the present invention provides a kit comprising a composition.

In another aspect, the present invention provides a kit, further comprising packaging and instructions for use.

In another aspect, the present invention provides a kit, wherein said packing comprises: a first container, wherein said first container contains 4-(3-(2H-1,2,3-triazol-2-yl)phenylamino)-2-((1R,2S)-2-aminocyclohexylamino)pyrimidine-5-carboxamide, or a pharmaceutically acceptable salt thereof; and a second container, wherein said second container contains said antineoplastic or anti-inflammatory agent.

In another aspect, the present invention provides a kit, further comprising a package insert stating that the two therapeutic agents can be used together.

The compositions of this invention are contemplated to provide for a synergistic effect in one or more of the following areas: improved therapeutic results, improved safety, reduced amount to achieve equivalent efficacy of one or more of the combination drugs as compared to the amount of that drug required to achieve the same level of efficacy when used alone.

These and other aspects, objects, features and advantages of the invention will be apparent upon reference to the following detailed description and figures. To this end, various references are set forth herein which describe in more detail certain background information, procedures, compounds and/or compositions, and are each hereby incorporated by reference in their entirety.

### BRIEF DESCRIPTION OF THE DRAWINGS

FIG. 1 shows the activity of 4-(3-(2H-1,2,3-triazol-2-yl)phenylamino)-2-((1R,2S)-2-aminocyclohexylamino)pyrimidine-5-carboxamide (Compound 1) in an RAT CIA model (Trentham et al., 1977) (Holmdahl et al., 2001) model of rheumatoid arthritis.

FIG. 2 shows dose responsive inhibitory activity of compound 1 and doxorubicin as single agents and when tested in combination. Viability of Diffuse Large B Cell Lymphoma cells (DHL4) are measured by quantitation of ATP via luminescence.

FIG. 3 shows analysis of combinatorial activity of compound 1 and doxorubicin. Isobologram depicting concentrations of inhibitors (compound 1 and doxorubicin) show expected concentrations (reference lines) which would be needed for additive activity leading to 50%, 70% or 90% inhibition. Synergistic activity of combination is established since lower concentration of individual agent (Compound 1 or doxorubicin alone) is required to attain same extent of inhibition.

FIG. 4 shows adhesion of rhodamine 6G labeled leukocytes to surface coated with collagen type I and JAM-C. Increased fluorescence is proportional to number of adherent cells under area of observation. Increased adherent cells in RA patients treated with antinflamamtory drugs (MTX, prednisone or anti TNF agent) depict inflammatory profile. In vitro addition of compound 1 reduced the number of adherent leukocytes.

FIG. 5 shows the kinetics of leukocyte recruitment/adhesion on collagen-JAMC in RA patients with compound 1.

FIG. 6 shows the kinetics of leukocyte recruitment was studied in whole blood from eight healthy volunteers and thirty RA patients. Data show that leukocytes from RA patients, (an inflammatory phenotype) have more adherent leukocytes in this assay system. In vitro addition of 3 uM Compound 1 (reduces the number of adherent cells. Since the RA patients are already being treated with therapeutic agents which are standard of care and still have the greater number of adherent cells than healthy controls; concomitant therapy with Compound 1 should lead to reduction of inflammatory state. Effect is seen in mild (n=8) FIG. 6A, moderate (n=14) FIG. 6B, and severe (n=10) FIG. 6C RA patients.

FIG. 7 shows the combination of prednisolone and Compound 1 can increase efficacy of subtherapeutic doses of each agent and produce statistically significant antiinflamamtory activity in mouse CAIA model. Literature reference for mouse model: The model has been described previously by (Hutamekalin et al., 2009, Terato et al., 1992).

FIG. 8 shows the combination of Compound 1 with MTX lowers target therapeutic level for immunomodulatory activity in RA patient blood. Syk inhibitory potency of compound 1 (PRT2607) was higher (reduced IC50) in blood from RA patients on stable doses of MTX. In vitro study of whole blood B cell activation (CD69) in stable RA patients treated with MTX (n=18) or without MTX (n=14). Compound 1 or vehicle control is added to whole blood from individual patients, lymphocytes (B cells) are activated by addition of anti IgD and activation marker (CD69) is quantitated by flow cytometry.

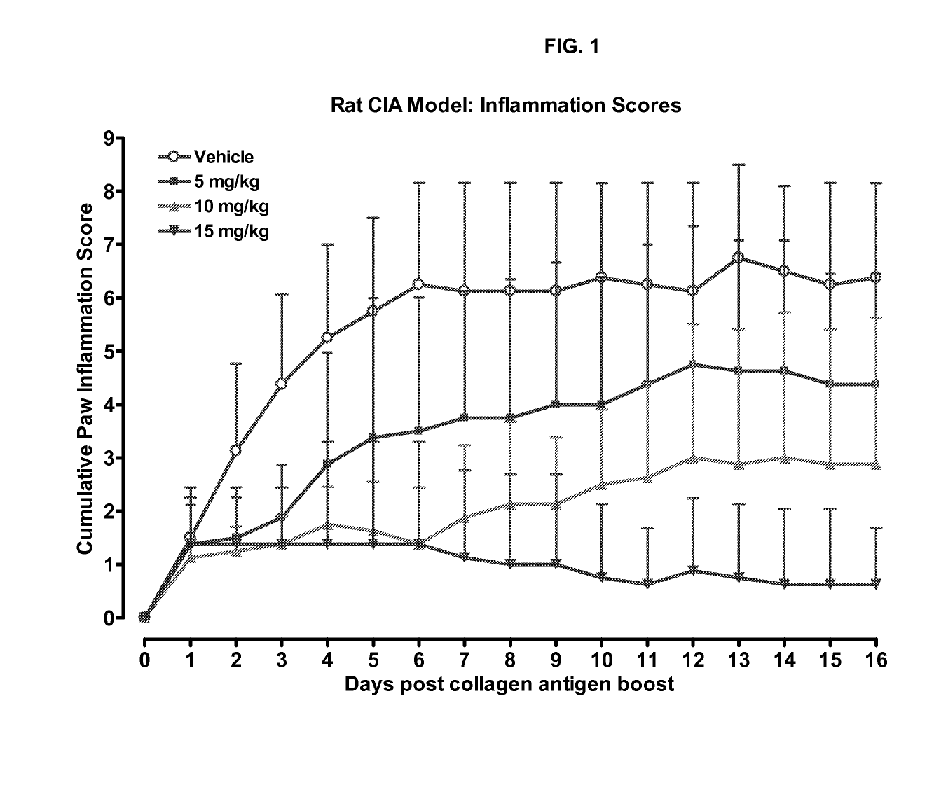

### DETAILED DESCRIPTION OF THE INVENTION

This invention relates to a method and compositions for treating syk-mediated conditions or disorders in a mammal using a combination of Compound 1 with a co-administered agent. Prior to describing this invention in more detail, the following terms are defined:

### 1. DEFINITIONS

It is noted here that as used in this specification and the appended claims, the singular forms “a,” “an,” and “the” include plural reference unless the context clearly dictates otherwise. Thus, for example, reference to “a pharmaceutically acceptable carrier” in a composition includes two or more pharmaceutically acceptable carriers, and so forth.

The term “administering” refers to oral administration, administration as a suppository, topical contact, intravenous (e.g. injected), intraperitoneal, intramuscular, intralesional, intranasal or subcutaneous administration, or the implantation of a slow-release device e.g., a mini-osmotic pump, to a subject. Adminsitration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Autoimmune disease” refers to generally include those disorders involving tissue injury that occurs as a result of a humoral and/or cell-mediated response to immunogens or antigens of endogenous and/or exogenous origin. Such diseases are frequently referred to as diseases involving the nonanaphylactic (i.e., Type II, Type III and/or Type IV) hypersensitivity reactions.

“Cell proliferative disease” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disease can have the same cell division rates as normal cells but do not respond to signals that limit such growth. Within the ambit of “cell proliferative disease” is neoplasm or tumor, which is an abnormal growth of tissue. Cancer refers to any of various malignant neoplasms characterized by the proliferation of cells that have the capability to invade surrounding tissue and/or metastasize to new colonization sites.

“Comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the term “composition” is intended to encompass a product comprising the specified ingredients in the specified amounts, as well as any product which results, directly or indirectly, from combination of the specified ingredients in the specified amounts.

The term “condition” refers to a disease state for which the methods and compositions of the present invention are being used against.

The term “inflammation” as used herein refers to infiltration of white blood cells (e.g., leukocytes, monocytes, etc.) into an area.

The term “leukocyte” refers to any of the various blood cells that have a nucleus and cytoplasm, separate into a thin white layer when whole blood is centrifuged, and help protect the body from infection and disease. Examples of leukocytes include, without limitation, neutrophils, eosinophils, basophils, lymphocytes, and monocytes.

The term “mammal” includes organisms which express syk. The term “mammal” includes, without limitation, human, bears, monkeys, rabbits, mice domestic animals, such as dogs and cats, farm animals, such as cows, horses, goats or pigs, and laboratory animals. Transgenic organisms which express syk are also included in this definition.

The terms “modulate”, “modulation” and the like refer to the ability of a compound to increase or decrease the function and/or expression of syk, where such function may include transcription regulatory activity and/or protein-binding. Modulation may occur in vitro or in vivo. Modulation, as described herein, includes the inhibition, antagonism, partial antagonism, activation, agonism or partial agonism of a function or characteristic associated with syk, either directly or indirectly, and/or the upregulation or downregulation of the expression of syk, either directly or indirectly. In a preferred embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction. The ability of a compound to inhibit the function of syk can be demonstrated in a biochemical assay, e.g., binding assay, or a cell-based assay, e.g., a transient transfection assay.

“Patient” refers to human and non-human animals, especially mammals. Examples of patients include, but are not limited to, humans, cows, dogs, cats, goats, sheep, pigs and rabbits.

The terms “pharmaceutically effective amount”, “therapeutically effective amount” or “therapeutically effective dose” refers to the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disorder or condition and its severity and the age, weight, etc., of the mammal to be treated.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M. et al., “Pharmaceutical Salts,” Journal of Pharmaceutical Science, 66:1-19, 1977). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Turning next to the compositions of the invention, the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. Pharmaceutically acceptable carriers” refer to any diluents, excipients, or carriers that may be used in the compositions of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The phrase “selectively” or “specifically” when referring to binding to a receptor, refers to a binding reaction that is determinative of the presence of the receptor, often in a heterogeneous population of receptors and other biologics. Thus, under designated conditions, the compounds bind to a particular receptor at least two times the background and more typically more than 10 to 100 times background. Specific binding of a compound under such conditions requires a compound that is selected for its specificity for a particular receptor. For example, small organic molecules can be screened to obtain only those compounds that specifically or selectively bind to a selected receptor and not with other receptors or proteins. A variety of assay formats may be used to select compounds that are selective for a particular receptor. For example, High-throughput screening assays are routinely used to select compounds that are selective for a particular a receptor.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

In some embodiments, it is contemplated that the therapeutically effective amount of Compound 1 or the co-administered agent in the combination can be less than their respective effective amount when used as a single agent. In this case, the therapeutically effective amount is referred to as “sub-therapeutic dosage.” Thus, the term “sub-therapeutic dosage” is intended to mean a dosage that is lower than the optimal dosage for a therapeutic agent when used as a single agent, but when used in the combinations described herein, provides a therapeutic result.

As used herein, the term “syk” refers to a spleen tyrosine kinase (RefSeq Accession No. P-043405) or a variant thereof that is capable of mediating a cellular response to T-cell receptors in vitro or in vivo. syk variants include proteins substantially homologous to native syk, i.e., proteins having one or more naturally or non-naturally occurring amino acid deletions, insertions or substitutions (e.g., syk derivatives, homologs and fragments). The amino acid sequence of syk variant preferably is at least about 80% identical to a native syk, more preferably at least about 90% identical, and most preferably at least about 95% identical.

The term “syk inhibitor” refers to any agent that inhibits the catalytic activity of spleen tyrosine kinase. Examples of known syk inhibitors include, without limitation, compounds described in U.S. Patent Publication Nos. 2010/0048567 and 2009/031840.

“Therapeutically effective amount” means an amount of Compound 1 or the co-administered agent of the present invention that is effective to treat a target disease or condition when administered in combination. The therapeutically effective amount will vary depending upon the specific combination, the subject and disease condition being treated, the weight and age of the subject, the severity of the disease condition, the dosing regimen to be followed, timing of administration, the manner of administration and the like, all of which can be determined readily by one of ordinary skill in the art.

The terms “treat”, “treating”, “treatment” and grammatical variations thereof as used herein, includes partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially.

The term “unit dosage form” refers to physically discrete units suitable as unitary dosages for human subjects and other mammals, each unit containing a predetermined quantity of drug calculated to produce the desired onset, tolerability, and/or therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of one or more syk inhibitors.

### 2. EMBODIMENTS OF THE INVENTION

### a. Compounds

U.S. Patent Publication No. 2010/0048567, titled “INHIBITORS OF SYK PROTEIN KINASE,” filed Apr. 16, 2009, the contents of which are incorporated herein by reference in its entirety, discloses a novel small-molecule SYK inhibitor compound, 4-(3-(2H-1,2,3-triazol-2-yl)phenylamino)-2-(1R,2S)-2-aminocyclohexylamino)pyrimidine-5-carboxamide, (Compound 1), which has the following structure:

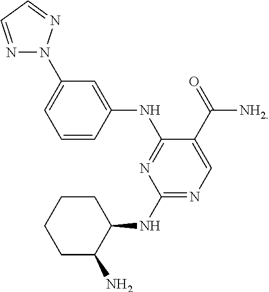 [SMILES: NC(=O)c1cnc(N[C@@H]2CCCC[C@@H]2N)nc1Nc1cccc(-n2nccn2)c1]

Compound 1 acts as a potent and selective inhibitor of SYK.

In one embodiment, the present invention provides a composition, wherein the pharmaceutically acceptable salt of 4-(3-(2H-1,2,3-triazol-2-yl)phenylamino)-2-(1R,2S)-2-aminocyclohexylamino)pyrimidine-5-carboxamide is the hydrochloride or acetate salt.

The neutral forms of the therapeutic agents may be regenerated by contacting the salt with a base or acid and isolating the parent therapeutic agent in the conventional manner. The parent form of the therapeutic agent differs from the various salt forms in certain physical properties, such as solubility in polar solvents, but otherwise the salts are equivalent to the parent form for the purposes of the present invention.

The compounds of the present invention may be prepared by known organic synthesis techniques, including the methods described in more detail in the Examples. It should also be noted that any heteroatom with unsatisfied valences in the text, schemes, examples and Tables herein is assumed to have the hydrogen atom to satisfy the valences.

### b. Inhibition of Syk Kinase

The activity of a specified combination of compounds may be assessed in vitro or in vivo. In some embodiments, the activity of a specified combination of compounds can be tested in a cellular assay. Exemplary assays of this type are described in greater detail in the Examples.

It must be further noted that the classification of certain therapeutic agents based on their intended use or mechanisms of action is based on the general knowledge of a person skilled in the art and for classification purposes only. The purported mechanisms are not intended to be used as a limitation for the therapeutic agents unless the context clearly dictates otherwise. Some therapeutic agents may act through two or more mechanisms or are able to be used to treat two or more conditions. It is also to be understood that the particular agents given in each categories are for examples only and are not intended to limit the scope of the present invention.

### c. Combination Therapy Methods and Pharmaceutical Compositions

The present invention further provides novel compositions comprising an agent selected from 4-(3-(2H-1,2,3-triazol-2-yl)phenylamino)-2-((1R,2S)-2-aminocyclohexylamino)pyrimidine-5-carboxamide, or a pharmaceutically acceptable salt thereof; and an antineoplastic or antiinflammatory agent.

It is contemplated that a combination of Compound 1 with a antineoplastic or antiinflammatory agent will produce additional anticancer or antiinflammatory effect over the two agents alone. As shown by Example 3, addition of varying amount of Compound 1 to a fixed amount of antineoplastic or antiinflammatory agent also produced additional decreased CLL viability in a dose responsive manner.

Not only can Compound 1 provide additive anticancer anticancer and/or anti-inflammatory benefit with an antineoplastic or antiinflammatory agent, it is also contemplated that Compound 1, an antineoplastic or antiinflammatory agent, can be combined with other classes of therapeutic agents to produce additional anticancer and/or anti-inflammatory benefit.

It is contemplated that the method of treatment using a combination of Compound 1 and a co-administered agent will not produce undesired drug-drug interaction or other additional side effects over the agents alone. Preferably, the combination can offer an improved efficacy and/or safety advantage over the agents alone, particularly when smaller dosing is required to achieve a therapeutic result. In such a case, the therapeutically effective amount of the agents in the combination therapy may be lower than the effective or optimal amount needed when the agents are used alone. It is contemplated that lower dosages will minimize potential side effects of an agent, thus lead to improved safety profile. Thus, the combination preferably allows one of the therapeutic agents to be used at a sub-therapeutic dosage. Still more preferably, the combination allows both therapeutic agents to be used at sub-therapeutic dosages.

Similarly, it is contemplated that the method of treatment using a combination of Compound 1, a antineoplastic or antiinflammatory agent and another therapeutic agent will not produce undesired drug-drug interaction or other additional side effects over use of any of the agents alone. Preferably the combination of three agents can offer an efficacy or safety advantage over the use of any of the agents alone. More preferably the combination allows one of the therapeutic agents be used at lower doses than that is required when the therapeutic agent is used alone, i.e. at sub-therapeutic dosages. Still more preferably, the combination allows all therapeutic agents to be used at sub-therapeutic dosages.

Compound 1 and the co-administered agent may be formulated into two separate pharmaceutical compositions. They may be administered at the same time or sequentially in any order. Preferably, when administered sequentially, the two agents are administered sufficiently closely in time so that the desired therapeutic effect can be provided. Compound 1 and the co-administered agent may also be formulated into a single pharmaceutical composition. Compound 1, an anticoagulant agent and another antiplatelet agent may also be administered at the same time or sequentially in any order. Preferably, when administered sequentially, the three agents are administered sufficiently closely in time so that the desired therapeutic effect can be provided. They may also be formulated into a single pharmaceutical composition or any two of them may be formulated into a single pharmaceutical composition.

Any of the above dosage forms containing effective amounts are within the bounds of routine experimentation and within the scope of the invention. A therapeutically effective dose may vary depending upon the route of administration and dosage form. The preferred combination of the invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals. Combination therapies of this invention may be administered once or several times daily and other dosage regimens may also be useful. Preferably, combination therapies of this invention are administered in a single daily dose, or administered two, three, or four times daily. More preferably, combination therapies of this invention are administered once or twice daily.

Typically, about 0.5 to 500 mg of Compound 1, or a salt or mixture of salts of Compound 1 is compounded with a physiologically acceptable vehicle, carrier, excipient, binder, preservative, stabilizer, dye, flavor etc., as called for by accepted pharmaceutical practice. In one aspect, Compound 1 is formulated into a formulation suitable for intravenous administration. In some embodiments, a unit dose of the intravenous formulation contains from 1 to 50 mg of Compound 1 or a pharmaceutically acceptable salt. In other embodiments, the unit dose contains from 5 to 40 mg, 10 to 30 mg, 15 to 25 mg, 25 to 45 mg, or about 20 mg, 30, 40, or 50 mg of Compound 1 or the salt.

In another aspect, Compound 1 is formulated into a formulation suitable for oral administration. In some embodiments, the composition is formulated as a unit dose containing from 1 to 800 mg, 20 to 500 mg, 30 to 250 mg, 40 to 200 mg, 50 to 150 mg, 100 to 120 mg, 10 to 50 mg, or 20 to 40 mg of Compound 1 or a salt. In some embodiments, the composition is in a unit dose format and contains about 30, 50, 55, 75, 90, 100, 110, 125, 150, 175, or 200 mg of Compound 1 or a salt.

When Compound 1 and fludarabine are formulated into a single pharmaceutical composition, about 0.5 to 500 mg of fludarabine can be added to the above composition. Preferably, when Compound 1 and fludarabine are formulated in an intravenous formulation, Compound 1 or a salt thereof is present in the amount of 1 to 50 mg, 5 to 40 mg, 10 to 30 mg, 15 to 25 mg, 25 to 45 mg, or about 20 mg, 30, 40, or 50 mg. When Compound 1 and fludarabine are formulated in an oral formulation, Compound 1 or a salt is present in the amount of from 1 to 800 mg, 20 to 500 mg, 30 to 250 mg, 40 to 200 mg, 50 to 150 mg, 100 to 120 mg, 10 to 50 mg, or 20 to 40 mg or about 30, 50, 55, 75, 90, 100, 110, 125, 150, 175, or 200 mg. In combinations containing Compound 1, any of the above unit doses of Compound 1 or a salt or mixture of salts of Compound 1 and about 0.5 to 500 mg of antineoplastic or antiinflammatory agent or a salt or mixture of salts of antineoplastic or antiinflammatory agent are compounded with a physiologically acceptable vehicle, carrier, excipient, binder, preservative, stabilizer, dye, flavor etc., as called for by accepted pharmaceutical practice. The amount of active ingredient(s) in these compositions is such that a suitable dosage in the range indicated is obtained.

It is contemplated that a concentration of Compound 1 in the combination therapies will range from about 0.001 μM to about 100 μM, preferably about 0.01 μM to about 50.0 μM, more preferably from about 0.01 μM to about 25.0 μM, and even more preferably from about 0.01 μM to about 10.0 μM. In combination therapies containing Compound 1 and antineoplastic or antiinflammatory agent, it is contemplated that a typical dosage of antineoplastic or antiinflammatory agent will range from about 0.001 μM to about 1000 μM, preferably from about 0.01 μM to about 10.0 μM, and more preferably from about 0.1 μM to about 1.0 μM.

It is contemplated that a typical dosage of Compound 1 in the combination therapies will range from about 0.001 mg/kg to about 100 mg/kg, preferably about 0.01 mg/kg to about 10.0 mg/kg, more preferably from about 0.01 mg/kg to about 2.0 mg/kg, and even more preferably from about 0.01 mg/kg to about 1.0 mg/kg. In combination therapies containing Compound 1 and antineoplastic or antiinflammatory agent, it is contemplated that a typical dosage of antineoplastic or antiinflammatory agent will range from about 0.001 mg/kg to about 1000 mg/kg, preferably from about 0.01 mg/kg to about 100.0 mg/kg, and more preferably from about 0.1 mg/kg to about 50 mg/kg, or from about 0.4 mg/kg to about 10 mg/kg, and even more preferably from about 0.5 mg/kg to about 5.0 mg/kg. Still more preferably, the dosage of antineoplastic or antiinflammatory agent in the combinations is lower than 1.0 mg/kg.

The typical dosages of the other co-administered agents described herein when used as a single agent are known to a person skilled in the art. It is contemplated that the dosages of these agents when used in combination with Compound 1 will not exceed the maximum dosages of the individual agents. Preferably, the dosages in the combination therapies are less than the maximum dosages and more preferably, the dosages in the combination therapies are sub-therapeutic dosages. It is contemplated that the dosages can be adjusted to reflect the improved benefit achieved by the combination therapies, which can be determined by one skilled in the art based on the information given herein.

The invention provides methods of treating a inflammatory or autoimmune disease selected from the group consisting of allergic reaction, transplant rejection, rheumatoid arthritis (RA), lupus, multiple sclerosis (MS) or psoriasis.

The invention provides methods of treating a cell proliferative disease selected from the group consisting of leukemia, a lymphoma, myeloproliferative disorders, hematological malignancies, and chronic idiopathic myelofibrosis.

In a specific embodiment, the compositions and methods can be used to treat these inflammatory, autoimmune and/or cell proliferative diseases in patients that are either initially non-responsive (resistant) to or that become non-responsive to treatment with one of the other current treatments for the particular disease. Suitable syk-inhibitory compounds with which the compounds can be administered are provided infra.

Generally, inflammatory and autoimmune diseases treatable with the compounds disclosed herein relate to any disease characterized by inflammation or hypersensitivity. These include allergic reaction, transplant rejection, rheumatoid arthritis (RA), lupus, multiple sclerosis (MS) or psoriasis.

Generally, cell proliferative diseases treatable with the compounds disclosed herein relate to any disorder characterized by aberrant cell proliferation. These include various tumors and cancers, benign or malignant, metastatic or non-metastatic. Specific properties of cancers, such as tissue invasiveness or metastasis, can be targeted using the methods described herein. Cell proliferative diseases include a variety of cancers, including, among others, ovarian cancer, renal cancer, gastrointestinal cancer, kidney cancer, bladder cancer, pancreatic cancer, lung squamous carcinoma, and adenocarcinoma.

In some embodiments, the cell proliferative disease treated is a hematopoietic neoplasm, which is aberrant growth of cells of the hematopoietic system. Hematopoietic malignancies can have its origins in pluripotent stem cells, multipotent progenitor cells, oligopotent committed progenitor cells, precursor cells, and terminally differentiated cells involved in hematopoiesis. Some hematological malignancies are believed to arise from hematopoietic stem cells, which have the ability for self renewal. For instance, cells capable of developing specific subtypes of acute myeloid leukemia (AML) (Hahn et al., Blood, 2007, 110, Abstract 209) upon transplantation display the cell surface markers of hematopoietic stem cells, implicating hematopoietic stem cells as the source of leukemic cells. Blast cells that do not have a cell marker characteristic of hematopoietic stem cells appear to be incapable of establishing tumors upon transplantation (Blaire et al., 1997, Blood 89:3104-3112). The stem cell origin of certain hematological malignancies also finds support in the observation that specific chromosomal abnormalities associated with particular types of leukemia can be found in normal cells of hematopoietic lineage as well as leukemic blast cells. For instance, the reciprocal translocation t(9q34;22q11) associated with approximately 95% of chronic myelogenous leukemia appears to be present in cells of the myeloid, erythroid, and lymphoid lineage, suggesting that the chromosomal aberration originates in hematopoietic stem cells. A subgroup of cells in certain types of CML displays the cell marker phenotype of hematopoietic stem cells.

Although hematopoietic neoplasms often originate from stem cells, committed progenitor cells or more terminally differentiated cells of a developmental lineage can also be the source of some leukemias. For example, forced expression of the fusion protein Bcr/Abl (associated with chronic myelogenous leukemia) in common myeloid progenitor or granulocyte/macrophage progenitor cells produces a leukemic-like condition. Moreover, some chromosomal aberrations associated with subtypes of leukemia are not found in the cell population with a marker phenotype of hematopoietic stem cells, but are found in a cell population displaying markers of a more differentiated state of the hematopoietic pathway (Turhan et al., 1995, Blood 85:2154-2161). Thus, while committed progenitor cells and other differentiated cells may have only a limited potential for cell division, leukemic cells may have acquired the ability to grow unregulated, in some instances mimicking the self-renewal characteristics of hematopoietic stem cells (Passegue et al., Proc. Natl. Acad. Sci. USA, 2003, 100:11842-9).

In some embodiments, the hematopoietic neoplasm treated is a lymphoid neoplasm, where the abnormal cells are derived from and/or display the characteristic phenotype of cells of the lymphoid lineage. Lymphoid neoplasms can be subdivided into B-cell neoplasms, T and NK-cell neoplasms, and Hodgkin lymphoma. B-cell neoplasms can be further subdivided into precursor B-cell neoplasm and mature/peripheral B-cell neoplasm. Exemplary B-cell neoplasms are precursor B-lymphoblastic leukemia/lymphoma (precursor B-cell acute lymphoblastic leukemia) while exemplary mature/peripheral B-cell neoplasms are B-cell chronic lymphocytic leukemia/small lymphocytic lymphoma, B-cell prolymphocytic leukemia, lymphoplasmacytic lymphoma, splenic marginal zone B-cell lymphoma, hairy cell leukemia, plasma cell myeloma/plasmacytoma, extranodal marginal zone B-cell lymphoma of MALT type, nodal marginal zone B-cell lymphoma, follicular lymphoma, mantle-cell lymphoma, diffuse large B-cell lymphoma, mediastinal large B-cell lymphoma, primary effusion lymphoma, and Burkitt's lymphoma/Burkitt cell leukemia.

T-cell and Nk-cell neoplasms are further subdivided into precursor T-cell neoplasm and mature (peripheral) T-cell neoplasms. Exemplary precursor T-cell neoplasm is precursor T-lymphoblastic lymphoma/leukemia (precursor T-cell acute lymphoblastic leukemia) while exemplary mature (peripheral) T-cell neoplasms are T-cell prolymphocytic leukemia T-cell granular lymphocytic leukemia, aggressive NK-cell leukemia, adult T-cell lymphoma/leukemia (HTLV-1), extranodal NK/T-cell lymphoma, nasal type, enteropathy-type T-cell lymphoma, hepatosplenic gamma-delta T-cell lymphoma, subcutaneous panniculitis-like T-cell lymphoma, Mycosis fungoides/Sezary syndrome, Anaplastic large-cell lymphoma, T/null cell, primary cutaneous type, Peripheral T-cell lymphoma, not otherwise characterized, Angioimmunoblastic T-cell lymphoma, Anaplastic large-cell lymphoma, T/null cell, primary systemic type. The third member of lymphoid neoplasms is Hodgkin lymphoma, also referred to as Hodgkin disease. Exemplary diagnosis of this class that can be treated with the compounds include, among others, nodular lymphocyte-predominant Hodgkin lymphoma, and various classical forms of Hodgkin disease, exemplary members of which are Nodular sclerosis Hodgkin lymphoma (grades 1 and 2), Lymphocyte-rich classical Hodgkin lymphoma, Mixed cellularity Hodgkin lymphoma, and Lymphocyte depletion Hodgkin lymphoma. In various embodiments, any of the lymphoid neoplasms that are associated with aberrant syk activity can be treated with Compound 1.

In some embodiments, the hematopoietic neoplasm treated is a myeloid neoplasm. This group comprises a large class of cell proliferative diseases involving or displaying the characteristic phenotype of the cells of the myeloid lineage. Myeloid neoplasms can be subdivided into myeloproliferative diseases, myelodysplastic/myeloproliferative diseases, myelodysplastic syndromes, and acute myeloid leukemias. Exemplary myeloproliferative diseases are chronic myelogenous leukemia (e.g., Philadelphia chromosome positive (t(9;22)(qq34;q11)), chronic neutrophilic leukemia, chronic eosinophilic leukemia/hypereosinophilic syndrome, chronic idiopathic myelofibrosis, polycythemia vera, and essential thrombocythemia. Exemplary myelodysplastic/myeloproliferative diseases are chronic myelomonocytic leukemia, atypical chronic myelogenous leukemia, and juvenile myelomonocytic leukemia. Exemplary myelodysplastic syndromes are refractory anemia, with ringed sideroblasts and without ringed sideroblasts, refractory cytopenia (myelodysplastic syndrome) with multilineage dysplasia, refractory anemia (myelodysplastic syndrome) with excess blasts, 5q-syndrome, and myelodysplastic syndrome. In various embodiments, any of the myeloid neoplasms that are associated with aberrant syk activity can be treated with Compound 1.

In some embodiments, the compounds can be used to treat Acute myeloid leukemias (AML), which represent a large class of myeloid neoplasms having its own subdivision of disorders. These subdivisions include, among others, AMLs with recurrent cytogenetic translocations, AML with multilineage dysplasia, and other AML not otherwise categorized. Exemplary AMLs with recurrent cytogenetic translocations include, among others, AML with t(8;21)(q22;q22), AML1(CBF-alpha)/ETO, Acute promyelocytic leukemia (AML with t(15;17)(q22;q11-12) and variants, PML/RAR-alpha), AML with abnormal bone marrow eosinophils (inv(16)(p13q22) or t(16;16)(p13;q11), CBFb/MYH11X), and AML with 11q23 (MLL) abnormalities. Exemplary AML with multilineage dysplasia are those that are associated with or without prior myelodysplastic syndrome. Other acute myeloid leukemias not classified within any definable group include, AML minimally differentiated, AML without maturation, AML with maturation, Acute myelomonocytic leukemia, Acute monocytic leukemia, Acute erythroid leukemia, Acute megakaryocytic leukemia, Acute basophilic leukemia, and Acute panmyelosis with myelofibrosis.

The inventive methods comprise administering an effective amount of a compound or composition described herein to a mammal or non-human animal. As used herein, “effective amount” of a compound or composition of the invention includes those amounts that antagonize or inhibit syk. An amount which antagonizes or inhibits syk is detectable, for example, by any assay capable of determining syk activity, including the one described below as an illustrative testing method. Effective amounts may also include those amounts which alleviate symptoms of a syk associated disorder treatable by inhibiting syk. Accordingly, “antagonists of syk” include compounds which interact with the syk, and modulate, e.g., inhibit or decrease, the ability of a second compound, e.g., another syk ligand, to interact with the syk. The syk binding compounds are preferably antagonists of syk. The language “syk binding compound” (e.g., exhibits binding affinity to the receptor) includes those compounds which interact with syk resulting in modulation of the activity of syk. Syk binding compounds may be identified using an in vitro (e.g., cell and non-cell based) or in vivo method. A description of in vitro methods are provided below.

The amount of compound present in the methods and compositions described herein should be sufficient to cause a detectable decrease in the severity of the disorder, as measured by any of the assays described in the examples. The amount of syk modulator needed will depend on the effectiveness of the modulator for the given cell type and the length of time required to treat the disorder. In certain embodiments, the compositions of this invention may further comprise another therapeutic agent.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, bioavailability modifiers and combinations of these.

Methods for preparing dosage forms are known to those skilled in the art (see, for example, REMINGTON'S PHARMACEUTICAL SCIENCES, 18TH ED., Mack Publishing Co., Easton, Pa. (1990)). In addition, pharmaceutically acceptable salts of Compound 1 of the present invention (e.g., acid addition salts) may be prepared and included in the compositions using standard procedures known to those skilled in the art of synthetic organic chemistry and described, e.g., by J. March, Advanced Organic Chemistry: Reactions, Mechanisms and Structure, 4^{th} Ed. (New York: Wiley-Interscience, 1992).

The compositions typically include a conventional pharmaceutical carrier or excipient and may additionally include other medicinal agents, carriers, adjuvants, diluents, tissue permeation enhancers, solubilizers, and the like. Preferably, the composition will contain about 0.01% to about 90%, preferably about 0.1% to about 75%, more preferably about 0.1% to 50%, still more preferably about 0.1% to 10% by weight of Compound 1, with the remainder consisting of suitable pharmaceutical carrier and/or excipients. Appropriate excipients can be tailored to the particular composition and route of administration by methods well known in the art, e.g., REMINGTON'S PHARMACEUTICAL SCIENCES, supra.

Pharmaceutically acceptable carriers that may be used in these compositions include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The compositions can additionally include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

Administration of a composition comprising Compound 1 with one or more suitable pharmaceutical excipients as advantageous can be carried out via any of the accepted modes of administration. Thus, administration can be, for example, oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. The formulations of the invention may be designed as short-acting, fast-releasing, or long-acting. Still further, compounds can be administered in a local rather than systemic means, such as administration (e.g., injection) as a sustained release formulation. According to a representative embodiment, the compositions of this invention are formulated for pharmaceutical administration to a mammal, preferably a human being.

The compositions of the present invention containing Compound 1 can be administered repeatedly, e.g., at least 2, 3, 4, 5, 6, 7, 8, or more times, or the composition may be administered by continuous infusion. Suitable sites of administration include, but are not limited to, skin, bronchial, gastrointestinal, anal, vaginal, eye, and ear. The formulations may take the form of solid, semi-solid, lyophilized powder, or liquid dosage forms, such as, for example, tablets, pills, capsules, powders, solutions, suspensions, emulsions, suppositories, retention enemas, creams, ointments, lotions, gels, aerosols, or the like, preferably in unit dosage forms suitable for simple administration of precise dosages.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The compositions of this invention are formulated for pharmaceutical administration to a mammal, preferably a human being. Such pharmaceutical compositions of the invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. The formulations of the invention may be designed as short-acting, fast-releasing, long-acting, sustained-releasing. Still further, compounds can be administered in a local rather than systemic means, such as administration (e.g., injection) as a sustained release formulation.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The pharmaceutical compositions of this invention may be in any orally acceptable dosage form, including capsules, tablets, aqueous suspensions or solutions. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be in a topical form, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative, such as benzylalkonium chloride. Alternatively, for ophthalmic uses, the pharmaceutical compositions may be formulated in an ointment, such as petrolatum.

The pharmaceutical compositions of this invention may also be administered by nasal aerosol or inhalation. Such compositions are prepared according to techniques known in the art of pharmaceutical formulation and may be prepared as solutions in saline, employing benzyl alcohol or other suitable preservatives, absorption promoters to enhance bioavailability, fluorocarbons and/or other conventional solubilizing or dispersing agents.

In addition to dosage forms described above, pharmaceutically acceptable excipients and carriers and dosage forms are generally known to those skilled in the art and are included in the invention. It should be understood that a specific dosage and treatment regimen for any particular patient will depend upon a variety of factors, including the activity of the specific compound employed, the age, body weight, general health, sex and diet, renal and hepatic function of the patient, and the time of administration, rate of excretion, drug combination, judgment of the treating physician or veterinarian and severity of the particular disease being treated. The amount of active ingredients will also depend upon the therapeutic agent combined with Compounds 1.

The pharmaceutical compositions of this invention may be in any orally acceptable dosage form, including tablets, capsules, cachets, emulsions, suspensions, solutions, syrups, elixirs, sprays, boluses, lozenges, powders, granules, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, the compositions take the form of a pill, tablet, or capsule, and thus, the composition can contain, along with Compound 1, a diluent such as lactose, sucrose, dicalcium phosphate, and the like; a disintegrant such as starch or derivatives thereof; a lubricant such as magnesium stearate and the like; and/or a binder such a starch, gum acacia, polyvinylpyrrolidone, gelatin, cellulose and derivatives thereof. A tablet can be made by any compression or molding process known to those of skill in the art. Compressed tablets may be prepared by compressing in a suitable machine Compound 1 in a free-flowing form, e.g., a powder or granules, optionally mixed with accessory ingredients, e.g., binders, lubricants, diluents, disintegrants, or dispersing agents. Molded tablets can be made by molding in a suitable machine a mixture of the powdered Compound 1 with any suitable carrier.

Alternatively, the pharmaceutical compositions of this invention may be in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax, polyethylene glycol (PEG), hard fat, and/or hydrogenated cocoglyceride. Compositions suitable for rectal administration may also comprise a rectal enema unit containing Compound 1 and pharmaceutically-acceptable vehicles (e.g., 50% aqueous ethanol or an aqueous salt solution) that are physiologically compatible with the rectum and/or colon. The rectal enema unit contains an applicator tip protected by an inert cover, preferably comprised of polyethylene, lubricated with a lubricant such as white petrolatum, and preferably protected by a one-way valve to prevent back-flow of the dispensed formula. The rectal enema unit is also of sufficient length, preferably two inches, to be inserted into the colon via the anus.

Liquid compositions can be prepared by dissolving or dispersing Compound 1 and optionally one or more pharmaceutically acceptable adjuvants in a carrier such as, for example, aqueous saline, aqueous dextrose, glycerol, ethanol, and the like, to form a solution or suspension, e.g., for oral, topical, or intravenous administration. Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The pharmaceutical compositions of this invention may also be in a topical form, especially when the target of treatment includes areas or organs readily accessible by topical application, including diseases of the eye, the skin, or the lower intestinal tract. Suitable topical formulations are readily prepared for each of these areas or organs. For topical administration, the composition containing Compound 1 can be in the form of emulsions, lotions, gels, foams, creams, jellies, solutions, suspensions, ointments, and transdermal patches.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyldodecanol, benzyl alcohol and water.

The pharmaceutical compositions of this invention may also be administered by nasal aerosol or inhalation. For delivery by inhalation, the compositions can be delivered as a dry powder or in liquid form via a nebulizer. Such compositions are prepared according to techniques known in the art of pharmaceutical formulation and may be prepared as solutions in saline, employing benzyl alcohol or other suitable preservatives, absorption promoters to enhance bioavailability, fluorocarbons and/or other conventional solubilizing or dispersing agents.

For ophthalmic use, the pharmaceutical compositions may be formulated as micronized suspensions in isotonic, pH adjusted sterile saline, or, preferably, as solutions in isotonic, pH adjusted sterile saline, either with our without a preservative, such as benzylalkonium chloride. Alternatively, for ophthalmic uses, the pharmaceutical compositions may be formulated in an ointment, such as petrolatum.

For parenteral administration, the compositions can be in the form of sterile injectable solutions and sterile packaged powders. Preferably, injectable solutions are formulated at a pH of about 4.5 to about 7.5.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The compositions of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

Any of the above dosage forms containing effective amounts are within the bounds of routine experimentation and within the scope of the invention. A therapeutically effective dose may vary depending upon the route of administration and dosage form. The representative compound or compounds of the invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

Besides those representative dosage forms described above, pharmaceutically acceptable excipients and carriers and dosage forms are generally known to those skilled in the art and are included in the invention. It should be understood that a specific dosage and treatment regimen for any particular patient will depend upon a variety of factors, including the activity of the specific compound employed, the age, body weight, general health, sex and diet of the patient, and the time of administration, rate of excretion, drug combination, judgment of the treating physician and severity of the particular disease being treated. The amount of active ingredient(s) will also depend upon the particular compound and other therapeutic agent, if present, in the composition.

### Kits

The invention further provides a novel kit or package wherein the inventive pharmaceutical compounds, compositions and/or salts thereof are used in combination with pharmaceutically acceptable carriers to treat states, disorders, symptoms and diseases where syk plays a role. In one aspect of this invention is to provide a kit comprising separate containers in a single package. In some embodiments, the kit of the present invention comprises: (a) a first container containing Compound 1 or pharmaceutically acceptable salt forms thereof, and (b) a second container containing an antineoplastic or antiinflammatory agent. In other embodiments, the kit comprises: (a) a first container containing Compound 1 or pharmaceutically acceptable salt forms thereof, (b) a second container containing an antineoplastic or antiinflammatory agent and (c) a third container containing another therapeutic agent. In some embodiments, the kit further contains a package insert stating that the two pharmaceutical agents can be used together for the treatment of inflammatory, autoimmune and cell proliferative diseases.

The first, second, or third container can be a bottle, jar, vial, flask, syringe, tube, bag, or any other container used in the manufacture, storage, or distribution of a pharmaceutical product. The package insert can be a label, tag, marker, or the like, that recites information relating to the pharmaceutical composition of the kit. The information recited will usually be determined by the regulatory agency governing the area in which the pharmaceutical composition is to be sold, such as the United States Food and Drug Administration. Preferably, the package insert specifically recites the indications for which the pharmaceutical composition has been approved. The package insert may be made of any material on which a person can read information contained therein or thereon. Preferably, the package insert is a printable material, such as paper, adhesive-backed paper cardboard, foil, or plastic, and the like, on which the desired information has been printed or applied.

### I. EXAMPLES

The following examples are offered to illustrate, but not to limit, the claimed invention.

The abbreviations used herein are conventional, unless otherwise defined. Unless stated otherwise, the following abbreviations used throughout the specification have the following meanings: DMSO=dimethyl sulfoxide; g=gram; HPLC=high performance liquid chromatography; hr=hour; kg=kilogram; L=liter; M=molar; mg=milligram; min=minute; mL=milliliter; mm=millimeter; ng=nanogram; nM=nanomolar; NMR=nuclear magnetic resonance; sec=second; THF=tetrahydrofuran; TLC=thin layer chromatography; μM=micromolar; μg=microgram.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1967-2004, Volumes 1-22; Rodd's Chemistry of Carbon Compounds, Elsevier Science Publishers, 1989, Volumes 1-5 and Supplementals; and Organic Reactions, Wiley & Sons: New York, 2005, Volumes 1-65.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range from about −78° C. to about 150° C., more preferably from about 0° C. to about 125° C., and most preferably and conveniently at about room (or ambient) temperature, e.g., about 20° C. to about 75° C.

Referring to the examples that follow, compounds of the present invention were synthesized using the methods described herein, or other methods, which are well known in the art.

The compounds and/or intermediates may be characterized by high performance liquid chromatography (HPLC) using a Waters Alliance chromatography system with a 2695 Separation Module (Milford, Mass.). The analytical columns may be C-18 SpeedROD RP-18E Columns from Merck KGaA (Darmstadt, Germany). Alternately, characterization may be performed using a Waters Unity (UPLC) system with Waters Acquity UPLC BEH C-18 2.1 mm×15 mm columns. A gradient elution may be used, typically starting with 5% acetonitrile/95% water and progressing to 95% acetonitrile over a period of 5 minutes for the Alliance system and 1 minute for the Acquity system. All solvents may contain 0.1% trifluoroacetic acid (TFA). Compounds may be detected by ultraviolet light (UV) absorption at either 220 or 254 nm. HPLC solvents may be from EMD Chemicals, Inc. (Gibbstown, N.J.). In some instances, purity may be assessed by thin layer chromatography (TLC) using glass backed silica gel plates, such as, for example, EMD Silica Gel 60 2.5 cm×7.5 cm plates. TLC results may be readily detected visually under ultraviolet light, or by employing well known iodine vapor and other various staining techniques.

Mass spectrometric analysis may be performed on one of two Agilent 1100 series LCMS instruments and the Acquity system with acetonitrile/water as the mobile phase. One system may use TFA as the modifier and measure in positive ion mode [reported as MH+, (M+1) or (M+H)+] and the other may use either formic acid or ammonium acetate and measure in both positive [reported as MH⁺, (M+1) or (M+H)⁺] and negative [reported as M−, (M−1) or (M−H)⁻] ion modes.

Nuclear magnetic resonance (NMR) analysis may be performed on some of the compounds with a Varian 400 MHz NMR (Palo Alto, Calif.). The spectral reference may be either TMS or the known chemical shift of the solvent.

The purity of some of the invention compounds may be assessed by elemental analysis (Robertson Microlit, Madison N.J.).

Melting points may be determined on a Laboratory Devices MeI-Temp apparatus (Holliston, Mass.).

Preparative separations may be carried out as needed, using either an Sq16x or an Sg100c chromatography system and prepackaged silica gel columns all purchased from Teledyne Isco, (Lincoln, Nebr.). Alternately, compounds and intermediates may be purified by flash column chromatography using silica gel (230-400 mesh) packing material, or by HPLC using a C-18 reversed phase column. Typical solvents employed for the Isco systems and flash column chromatography may be dichloromethane, methanol, ethyl acetate, hexane, acetone, aqueous hydroxyamine and triethyl amine. Typical solvents employed for the reverse phase HPLC may be varying concentrations of acetonitrile and water with 0.1% trifluoroacetic acid.

### General Methods

The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

### Example 1

### 4-(3-(1H-1,2,3-triazol-2-yl)phenylamino)-2-((1R,2S)-2-aminocyclohexylamino)pyrimidine-5-carboxamide and hydrochloride (Compound 1)

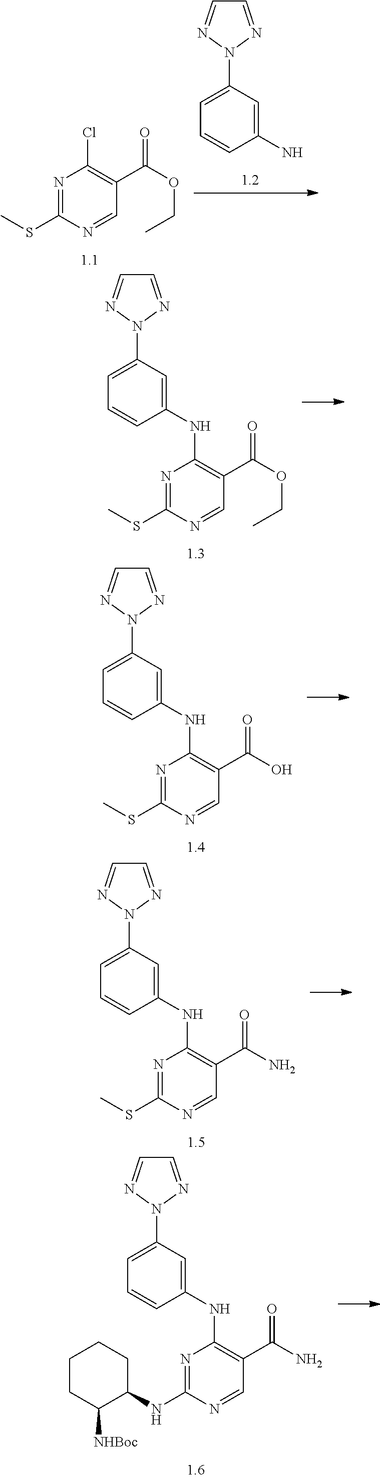

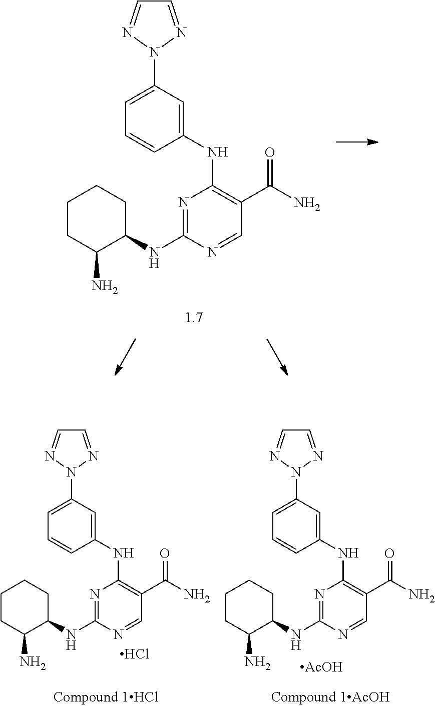

Step 1: Ethyl-4-chloro-2-methylthio-5-pyrimidine carboxylate 1.1 (1752 g) and ethanol (8600 ml) were charge to the vessel under nitrogen. Triethylamine (790 g) was added to the mixture dropwise. An exotherm of 2° C. was observed during the addition. Triazole aniline 1.2 (1210 g) was charged to the vessel in portions. Initially an endotherm of 3° C. was observed, however after ˜1 h the reaction temperature had raised by 5° C. and a white precipitate had formed. After stirring the reaction for 17 h HPLC analysis indicated 0.54% pyrimidine staring material remaining. Water (26.3 L) was charged to the vessel and the slurry was stirred for 0.5 h. The solids were collected by filtration, washed with water (2×8.8 L) and dried in vacuo at 40° C. for 120 h, yielding 2505 g (93%) of compound 1.3 of purity 97% by HPLC as a light yellow solid. MS found for C₁₆H₁₆N₆O₂S as (M+H)⁺ 356.1.

Step 2: Ethyl Ester 1.3 from Step 1 (2505 g) was charged to a 50 L vessel under nitrogen. THF (4800 ml) was charged to the vessel and the stirrer was started. A solution of lithium hydroxide monohydrate (320 g) in water (4584 ml) was charged to the vessel dropwise. The reaction was stirred for 17 h after which HPLC analysis indicated 1.1% stage 1 remaining. A further 1.1% (3.5 g) of the original charge of lithium hydroxide monohydrate was charged to the vessel. After stirring for a further 24 h the reaction was deemed complete with 0.48% stage 1 remaining by HPLC. The reaction was cooled to between 5-10° C. and taken to pH 1 with 1M HCl (10 L). The reaction mixture was diluted with THF:water (1:1, 18.5 L) and allowed to stir for 0.5 h at 10° C. The solid was isolated by filtration and washed with water (4.5 L×7) until the filtrate was pH 7. The material was dried in vacuo at 50° C. to yield a total of 2045.6 g (97.1%) carboxylic acid intermediate 1.4 with purity >99% by HPLC and >95% by ¹H NMR. MS found for C₁₅H₁₃N₅O₂S as (M+H)⁺ 328.1.

Step 3: Carboxylic acid 1.4 (1392.4 g) and anhydrous DMF (27.8 L) was charged to a 50 L vessel under nitrogen. CDI (1011.4 g) was added to the vessel portion wise. After the addition, complete dissolution occurred. The reaction was stirred at 20-25° C. for 2 h after which HPLC analysis indicated 0.4% stage 2 and 97.9% CDI intermediate. The reaction solution was transferred to 37% aqueous ammonia solution (14.0 L) whilst stirring and the viscous slurry was stirred at 20° C. for 16 h. After the stir out HPLC analysis indicated 0.07% CDI intermediate with 98.4% step 3. The reaction mixture was quenched into water (28.0 L) precipitating out the product. The mixture was stirred for 0.5 h. The solid was isolated by filtration, washed with water (2×7.0 L) and dried in vacuo at 50° C. for 120 h to yield 1357.3 g (97.8%) amide compound 1.5 of purity 99.43% by HPLC and >95% by ¹H NMR. MS found for C₁₅H₁₄N₆OS as (M+H)⁺ 327.1.

Step 4: Under N₂ was charged stage 3 (1200.0 g) and 1-methyl-2-pyrrolidinone (12000 mL). The resulting solution was cooled to 4° C. and 70% 3-chloroperoxybenzoic acid (1349.7 g) was added portionwise over 35 minutes with a final temperature of 8° C. observed. The reaction mixture was warmed to 20-25° C. and allowed to stir at this temperature overnight. HPLC analysis indicated 0.83% stage 3 remaining (target <2.0%). A solution of the tert-Butyl (1S,2R)-2-aminocyclohexylcarbamate (chiral diamine, 825.8 g) in N,N-diisopropylethyl-amine (1900 mL) was added dropwise over 16 minutes, maintaining the temperature between 20-30° C. The reaction mixture was heated to 75-85° C. and left to stir for 3 h. HPLC analysis indicated 82.9% stage 4 (target >75%). The reaction mixture was cooled to 20° C. and polish filtered through a sinter. The vessel was cleaned and approximately half the filtrate (7 L) was charged back into the vessel. Polish filtered purified water (12000 mL) was charged to the vessel and the temperature was adjusted to 38° C. Polish filtered ethyl acetate (5400 mL) was charged and allowed to stir between 35-40° C. for 10 minutes. The layers were separated and the aqueous was extracted with polish filtered ethyl acetate (2×5400 mL). The organic layers were combined and concentrated at 40° C. The remaining filtrate (9 L) was charged back into the vessel. Polish filtered purified water (12000 mL) was charged to the vessel and the temperature was adjusted to 38° C. Polish filtered ethyl acetate (5400 mL) was charged and allowed to stir between 35-40° C. for 10 minutes. The layers were separated and the aqueous was extracted with polish filtered ethyl acetate (2×5400 mL). The organic layers were combined and concentrated at 40° C. to give 3970.0 g crude material. Polish filtered water (24000 mL) was charged to the vessel and warmed to 25° C. The crude product was charged dropwise over 3 h to form an off-white suspension. The reaction mixture was stirred for 30 minutes between 25-30° C. before collecting the solid via vacuum filtration and washing with purified water (6000 mL). The solid was dried, under vacuum at 50° C. to yield 1700.2 g (94.0%). MS found for C₂₄H₃₁N₉O₃ as (M+H)⁺ 494.2.

Step 7: To a solution of 3M HCl in EtOAc (300 mL) was charged Compound 1.6 (0.47 g) portionwise. The suspension was stirred at rt until complete deprotection was observed by HPLC. EtOH (12 ml) was added to the reaction mixture and cooled to 5-10° C. The suspension was basified with 5M aq. NaOH (17 mL) to pH 8/9 and water (15 mL) was charged. The layers separated and the aqueous layer was extracted with EtOAc (2×50 mL). The combined organic layers were evaporated and the residue azeotroped with EtOAc (3×50 mL). The solid product was dried under vacuum at 50° C. to give 0.37 g of Compound 1.7 as free base as a pale yellow solid (97.6% purity by NMR).

### Compound 1 HCl salt synthesis, 4-(3-(2H-1,2,3-triazol-2-yl)phenylamino)-2-((1R,2S)-2-aminocyclohexylamino)pyrimidine-5-carboxamide hydrochloride

To a suspension of Compound 1.7 (free base, 1.16 g, 2.54 mol) in ethanol (25 ml) was added 4M HCl in dioxane (3.2 ml) over 5 mins. The reaction mixture was stirred at 40° C. for 3.5 hrs then cooled to 30° C. and filtered with washing of the filter cake (Et₂O 15 ml). The resulting solids were dried at 45° C. under vacuum to give 4-(3-(2H-1,2,3-triazol-2-yl)phenylamino)-2-((1R,2S)-2-aminocyclohexylamino)pyrimidine-5-carboxamide as a hydrochloride salt (Compound 1 HCl) in 0.79 g, 85% yield. LCMS indicates >98% purity. ¹H NMR and MS corresponds to the desired product. MS found for C₂₀H₂₄N₈O as MS found for C₁₉H₂₃N₉O as (M+H)⁺ 394.3. UV λ=250 nm. NMR (DMSO-d6): δ 12.4 (s, 1H), 8.89 (s, 1H), 8.82 (s, 1H), 8.60 (s, 1H), 8.36-8.06 (m, 5H), 7.90 (s, 1H), 7.83 (d, J=7.6 Hz, 1H), 7.45 (dd, J=8.4, 8.0 Hz, 1H), 7.23 (d, J=8.0 Hz, 1H), 4.52 (m, 1H), 3.58 (m, 1H), 1.82-1.43 (m, 8H) ppm.

### Compound 1 acetate salt synthesis, 4-(3-(2H-1,2,3-triazol-2-yl)phenylamino)-2-((1R,2S)-2-aminocyclohexylamino)pyrimidine-5-carboxamide acetate

Under N₂ was charged Compound 1.7 (free base, 2855.9 g), polish filtered ethanol (26280 mL) and polish filtered water (2290 mL). The suspension was heated to 47° C. and polish filtered acetic acid (620 mL) was added in one portion. The reaction mixture was stirred between 45-50° C. for 30 minutes before heating to reflux (79° C.) and stirring for 30 minutes. The suspension was cooled to 20° C. and the solid was collected via vacuum filtration and washed with ethanol (3×4860 mL) and t-butylmethylether (3×4860 mL). A damp yield of 2754.8 g was dried in vacuo at 50° C. overnight to afford Compound 1 as acetate salt, 4-(3-(2H-1,2,3-triazol-2-yl)phenylamino)-2-((1R,2S)-2-aminocyclohexylamino)pyrimidine-5-carboxamide acetate salt as a pale yellow solid (Yield=2120.9 g, 64.4%). MS found for C₂₀H₂₄N₈O as MS found for C₁₉H₂₃N₉O as (M+H)⁺ 394.3. UV λ=250 nm. NMR (DMSO-d6): δ 12.00 (s, 1H), 8.93 (s, 1H), 8.70 (s, 1H), 8.18-8.04 (s, 2H), 7.71 (d, J=7.6 Hz, 1H), 7.55 (dd, J=8.4, 8.0 Hz, 1H), 7.44 (d, J=8.0 Hz, 2H), 5.80-5.56 (m, 4H), 4.15 (m, 1H), 3.27-3.08 (m, 1H), 2.58-2.38 (s, 1H), 1.88-1.81 (s, 3H, acetate), 1.81-1.40 (m, 6H), 1.40-1.13 (m, 2H) ppm.

### Example 3

### Combination of Compound 1 and Antineoplastic or Antiinflammatory Agent in a Rodent Model of Rheumatoid Arthritis

Data are all derived from rat model of CIA.

### Description of Model:

### Rat Collagen Induced Arthritis (CIA) Model

The effects of methotrexate or dexamethasone or Compound 1 treatment was investigated in a severe model of joint inflammation. A collagen induced arthritis (CIA) was induced in female, 7-week old, Lewis rats by subcutaneous injection of bovine collagen II (Chondrex, Inc.) emulsified with incomplete Freund's adjuvant at the base of the tail (Trentham D E, Townes A S, Kang A H. Autoimmunity to type II collagen an experimental model of arthritis. J Exp Med 1977; 146:857-68.

Holmdahl R, Lorentzen J C, Lu S, Olofsson P, Wester L, Holmberg J, et al. Arthritis in rats with non-immunogenic adjuvants as models for rheumatoid arthritis. Immunol Rev 2001; 184:184-202). On day 10, the rats were boosted with a second subcutaneous injection. Test article administration was initiated when at least one hind paw was inflamed (clinical arthritis score=1) and enrollment into a treatment group was designated as day 1 of the study for each individual animal (n=4 to 8/dose group). Progression of disease was evidenced by increased edema and erythema of one or both ankle joints, followed by the involvement of the metatarsal and interphalangeal joints. Fully developed arthritis was observed approximately 4-7 days after the onset of inflammation. Hind paws were scored daily (inflammation score) for progression of disease and evaluated for histopathological changes in cartilage structure, cellularity, matrix glycoaminoglycans, and synovial inflammation at the end of the 16-day treatment course. The model is considered appropriate for mimicking inflammatory diseases as the anti-collagen antibodies are endogenously produced, the resulting inflammation produces a severe and irreversible arthritic injury (ankylosis) of the hind ankle joint and instead of prophylactic treatment, test article is started after demonstration of hind paw inflammation. In Tables below, mean scores are calculated for all animals in dosed group over the entire experimental time course.

### Experimental Data for Methotrexate Treatment

| | | Metho- | Metho- | Metho- | Metho- |
|---|---|---|---|---|---|
| | | trexate | trexate | trexate | trexate |
| | Vehicle | 0.1 mg/kg | 0.3 mg/kg | 1.0 mg/kg | 2.0 mg/kg |
| Number of | 16 | 16 | 16 | 16 | 16 |
| replicates | | | | | |
| Minimum | 1.80 | 1.25 | 1.00 | 1.13 | 1.00 |
| Inflammation | | | | | |
| Score | | | | | |
| measurement | | | | | |
| Maximum | 6.25 | 4.63 | 5.00 | 4.00 | 2.50 |
| Inflammation | | | | | |
| Score | | | | | |
| measurement | | | | | |
| Mean | 5.10 | 3.65 | 4.02 | 2.90 | 2.11 |
| Inflammation | | | | | |
| Score | | | | | |
| Std. Deviation | 1.42 | 1.01 | 1.35 | 0.94 | 0.47 |
| Std. Error | 0.502 | 0.357 | 0.477 | 0.332 | 0.166 |

### Experimental Data for Compound 1 Treatment (Two Separate Experimental Designs)

| | | Compound 1 | Compound 1 | Compound 1 |
|---|---|---|---|---|
| | Vehicle | 5 mg/kg | 15 mg/kg | 50 mg/kg |
| Number of | 16 | 16 | 16 | 16 |
| replicates | | | | |
| Minimum | 1.125 | 1.5 | 0.75 | 0 |
| Inflammation | | | | |
| Score | | | | |
| measurement | | | | |
| Maximum | 6.375 | 5.625 | 1.125 | 1.0 |
| Inflammation | | | | |
| Score | | | | |
| measurement | | | | |
| Mean | 5.287 | 4.868 | 0.949 | 0.169 |
| Inflammation | | | | |
| Score | | | | |
| Std. Deviation | 1.411 | 1.079 | 0.153 | 0.261 |
| Std. Error | 0.499 | 0.381 | 0.054 | 0.092 |

| | | Compound 1 | Compound 1 | Compound 1 |
|---|---|---|---|---|
| | Vehicle | 5 mg/kg | 10 mg/kg | 15 mg/kg |
| Number of | 16 | 16 | 16 | 16 |
| replicates | | | | |
| Minimum | 1.50 | 1.38 | 1.13 | 0.63 |
| Inflammation | | | | |
| Score | | | | |
| measurement | | | | |
| Maximum | 6.75 | 4.75 | 3.00 | 1.38 |
| Inflammation | | | | |
| Score | | | | |
| measurement | | | | |
| Mean | 5.58 | 3.57 | 2.15 | 1.016 |
| Inflammation | | | | |
| Score | | | | |
| Std. Deviation | 1.42 | 1.11 | 0.68 | 0.32 |
| Std. Error | 0.502 | 0.392 | 0.240 | 0.113 |

### Experimental Data for Dexamethasone Treatment

| | | Dexamethasone |
|---|---|---|
| | Vehicle | 1 mg/Kg |
| Number of replicates | 14 | 8 |
| Minimum Inflammation | 1.57 | 0.0 |
| Score measurement | | |
| Maximum Inflammation | 5.71 | 1.50 |
| Score measurement | | |
| Mean Inflammation | 4.60 | 0.188 |
| Score | | |
| Std. Deviation | 1.20 | 0.393 |
| Std. Error | 0.453 | 0.197 |

TABLE
Lymphocyte Counts in rats with bovine
collagen antibody mediated inflammation.

| | Vehicle Control | Dexamethasone 1 mg/kg |
|---|---|---|
| Animal # | (×10⁻³) | (×10⁻³) |
| 1 | 4.52 | 0.72 |
| 2 | 3.43 | 5.94 |
| 3 | 2.47 | 1.36 |
| 4 | 1.84 | 1.26 |
| 5 | 3.57 | |
| 6 | 4.68 | |
| 7 | 1.99 | |

In 7 control rats treated with vehicle, lymphocyte count was 3.21×103/μt. In 4 rats treated with dexamethasone, count=2.32×103/μL, p=0.42. However 3 out of 4 of rats did suffer from lymphopenia. Since Compound 1 does not produce a reduction in lymphocyte counts when administered as a single agent, a combination of reduced dose Dex+Compound 1 will provide an opportunity to produced therapeutic benefit without immunecompromising effect of drop in lymphocyte blood cell count.

Similarly, other antineoplastic agents or antiinflammatories (e.g., methotrexate, dexamethazone, prednisone, cyclophosphamide/cytoxan, adriamycin/doxorubicin/hydroxorubicin/danorubicin, proteosome inhibitors such as bortezomib, HDAC inhibitors, bleomycin, vincristine, doxorubicin, ifosfamide, carboplatin, etoposide, cytarabine, cisplatin, Mitoxantrone, Pralatrexate (Folotyn), chlorambucil, bendamustine, gemcitabine, Revlimid, thalidomide and analogs, Pentostatin, cladribine, Allopurinol, Alemtuzumab (Campath-1H), Oxaliplatin, cytarabine, rituximab, Flavopiridol, mitoxantrone, anti-CD20 antibodies such as ofatumumab (HuMax CD20), Lenalidomide, Nelarabine and Navelbine; and the like) are expected to demonstrate significant decreases in cell viability in CLL cells in this anti-leukemia model. It is contemplated that when non-effective doses of methotrexate, dexamethazone, prednisone, cyclophosphamide/cytoxan, adriamycin/doxorubicin/hydroxorubicin/danorubicin, proteosome inhibitors such as bortezomib, HDAC inhibitors, bleomycin, vincristine, doxorubicin, ifosfamide, carboplatin, etoposide, cytarabine, cisplatin, Mitoxantrone, Pralatrexate (Folotyn), chlorambucil, bendamustine, gemcitabine, Revlimid, thalidomide and analogs, Pentostatin, cladribine, Allopurinol, Alemtuzumab (Campath-1H), Oxaliplatin, cytarabine, rituximab, Flavopiridol, mitoxantrone, anti-CD20 antibodies such as ofatumumab (HuMax CD20), Lenalidomide, Nelarabine and Navelbine; and the like or other antineoplastic agents or antiinflammatories as described herein are combined with non-effective doses of Compound 1, potent synergistic decreases in leukemic cell viability will be observed.

### Example 4

### Combination of Compound 1 and Antineoplastic or Antiinflammatory Agent in a NHL Model

The following table gives activity of Compound 1 in NHL cell lines.

TABLE
Activity of Compound 1 in NHL Cell Lines.

| | SU- | SU- | | Toledo (SYK |
|---|---|---|---|---|
| | DHL4 | DHL6 | Ramos | independent) |
| BCR-induced Syk | 160 to | | | |
| (Y525/526) auto- | 400 nM | | | |
| phosphorylation | | | | |
| (IC50) | | | | |
| BCR-induced pBLNK | 160 nM | 10 to | | |
| formation (IC50 ) | | 20nM | | |
| BCR-induced pERK | | 0.1 | 0.05-0.125 | |
| formation (IC50 μM) | | | | |
| BCR-induced pAKT | | | 0.05-0.125 | |
| formation (IC50 μM) | | | | |
| Intracellular calcium | | 0.111 | 0.117 | |
| flux (IC50 μM) | | | | |
| Cell proliferation — | 1.8 | 1.1 | | 9.3 |
| MTT assay (IC50 μM) | | | | |
| Induction of apoptosis— | 15.5 | 20.3 | | 2.9 |
| Annexin V binding | | | | |
| (% +ve cells) | | | | |
| Induction of apoptosis— | 26 | 47 | | 0 |
| caspase 3 cleavage | | | | |
| (% +ve cells) | | | | |

The following table gives ex vivo activity of Compound 1 added to blood from healthy normal volunteers. Sub micromolar potency of inhibition of BCR signaling (pERK) and activation (CD69) shows ability to inhibit pathways implicated in survival of B Cell lymphoma cells and leukemia cells.

TABLE
B Cell activity in human whole blood.

| ASSAY | IC50 (μM) |
|---|---|
| BCR-induced pERK formation | 0.25-0.5 |
| BCR-induced CD69 expression | 0.25-0.5 |

### Example 5

### Synergistic Activity of Compound 1 with Doxorubicin in Diffuse Large B Cell Lymphoma Cell Lines

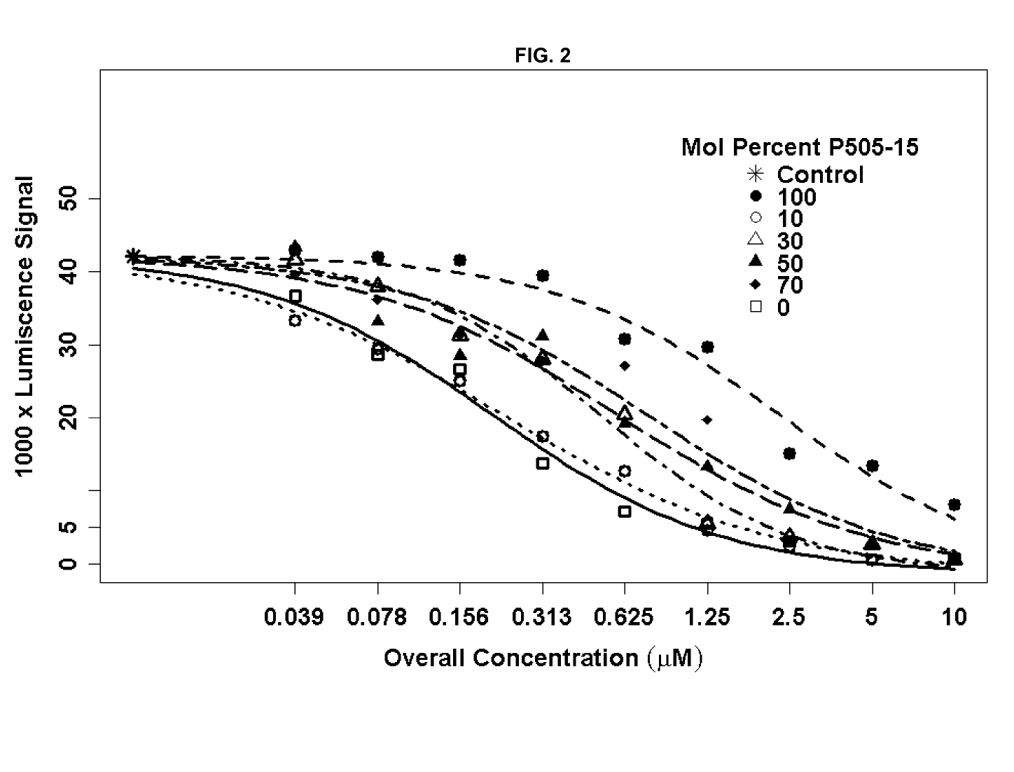

Compound 1, doxorubicin (Sigma) or mixtures of both at constant ratios were serially diluted in DMSO in a 96 well round bottom plate and aliquots were transferred to second 96 well plate with RPMI media. 8 μL of the final dilutions were then pipetted into a 384 well assay plate (Corning 3570) and 32 μL of the DLBCL cell line DHL4 was added at 5,000 cells per well in RPMI media containing 10% fetal calf serum. The final concentration of solvent (DMSO) during experimental time course was 1%. The robotic system Biomek 2 was used for all pipetting steps. After three days at 37 degrees in a tissue culture incubator, viability of cells was determined by lysis and luminescent ATP detection. ATP detection was carried out by addition of 20 μL Cell Titer Glo (Promega) following the protocol provided by manufacturer. Data analysis was done using DRC package of R software (Ritz C and Streibig J. Bioassay analysis using R, J Stat Softw 2005; 12: 1-22) (see FIG. 2).

### Example 6

### Synergistic Activity of Compound 1 with Doxorubicin in Diffuse Large B Cell Lymphoma Cell Line

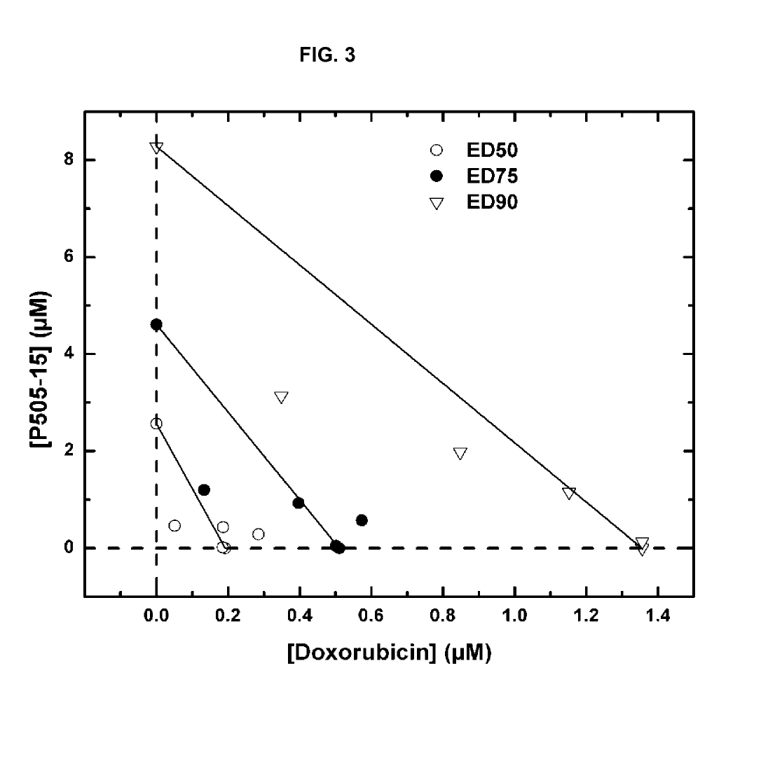

ED50, ED75 and ED75 for doxorubicin, Compound 1 and several fixed ratio combinations of both were evaluated for the inhibition of cell growth of the lymphoma cell line DHL4 using the dose-effect plots of Chou-Talalay (Chou T C. Drug combination studies and their synergy quantification using the Chou-Talalay method. Cancer Res. 2010; 70(2):440-446). The concentrations of the inhibitors required to obtain 50, 75 or 90 percent inhibition are plotted in an isobologram (Tallarida R J. Dose response relations in pharmacology, Chapman and Hall/CRC, 2000). Additive effects are marked by reference lines connecting the ED50, ED75 and ED90 values of the individual compounds (Compound 1 and doxorubicin). Since synergism reduces the concentration of compound necessary for inhibition, the corresponding ED50, ED75 and ED90 of the mixture appear below the line (see FIG. 3).

### Example 7

### Activity of Compound 1 and Doxorubicin in Burkitt's Lymphoma Cell Lines

Compound 1 or doxorubicin (Sigma) was serially diluted in DMSO in a 96 well round bottom plate and aliquots were transferred to a second 96 well plate with RPMI media. 8 μL of the final dilutions were then transferred into a 384 well assay plate (Corning 3570) and 32 μL of the Burkitt's lymphoma cell lines (Ramos or Daudi) were added at 5,000 cells per well in RPMI media containing 10% fetal calf serum. The final concentration of solvent (DMSO) during experimental time course was 1%. The robotic system Biomek 2 was used for all liquid handling steps. After three days at 37 degrees in a tissue culture incubator, viability of cells was determined by lysis and luminescent ATP detection. ATP detection was carried out by addition of 20 μL Cell Titer Glo (Promega) following the protocol provided by manufacturer. Data from two independent determinations of Compound 1 are presented in table below. Each determination was carried out in four replicates per plate. Data analysis was done using DRC package of R software (Ritz, C and Streibig, J Bioassay analysis using R, J Stat Softw 2005; 12: 1-22).

| Cell Line | Agent | ED50 | Hill slope |
|---|---|---|---|
| Ramos | Compound 1 | 1.05, 1.09 | 1.17, 1.43 |
| | Doxorubicin | 0.22 | 3.7 |
| Daudi | Compound 1 | 0.52, 0.36 | 1.42, 1.44 |
| | Doxorubicin | 0.1 | 1.26 |

### Example 8

### Leukocyte Rolling and Adhesion in Whole Blood from Patients with Rheumatoid Arthritis is Inhibited by Compound 1

Junction adhesion molecule C (JAM-C) mediates recruitment and adhesion of leukocytes to synovium of patients with rheumatoid arthritis (Rabquer B J, Arthritis Rheum, 2008; 58(10):3020-9). This is an important step in the etiology of rheumatoid arthritis and disruption of this activity may have therapeutic benefit in the treatment of patients with inflammatory diseases such as RA. In order to use an in-vitro experimental system to mimic migration and adhesion of leukocytes, we have used a collagen and JAM-C coated surface for the assay.

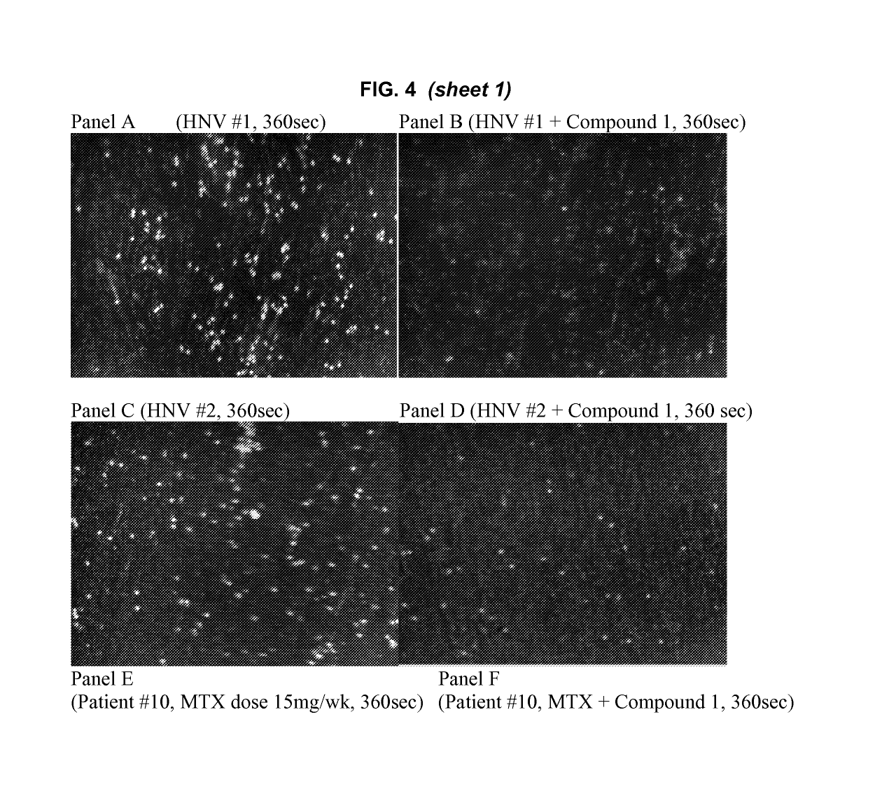

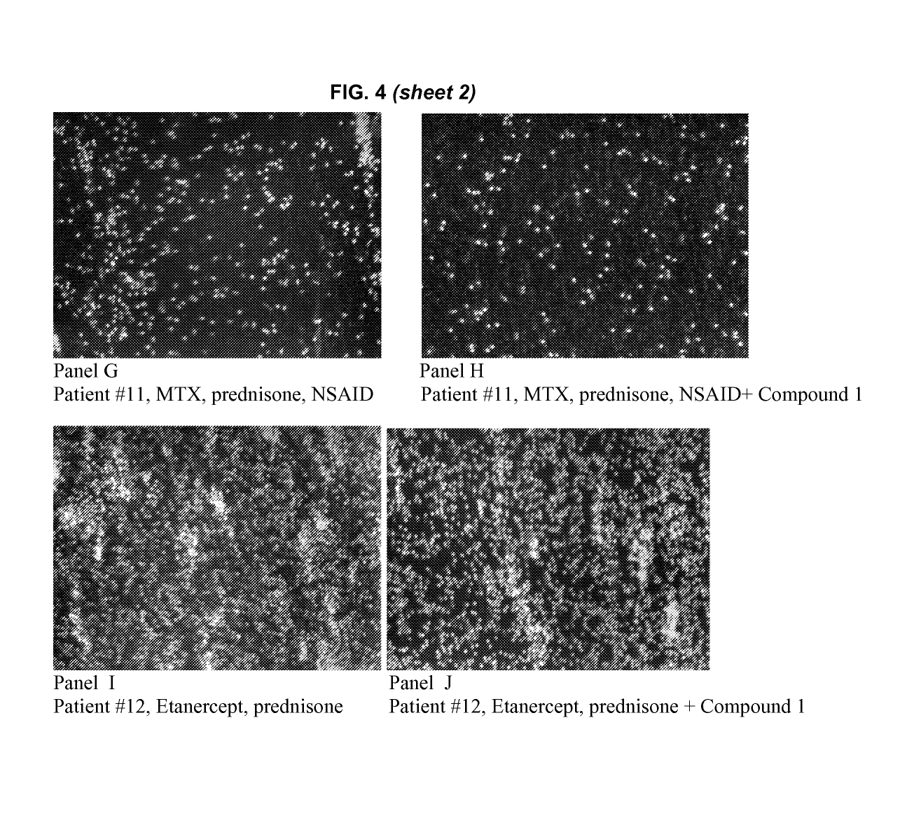

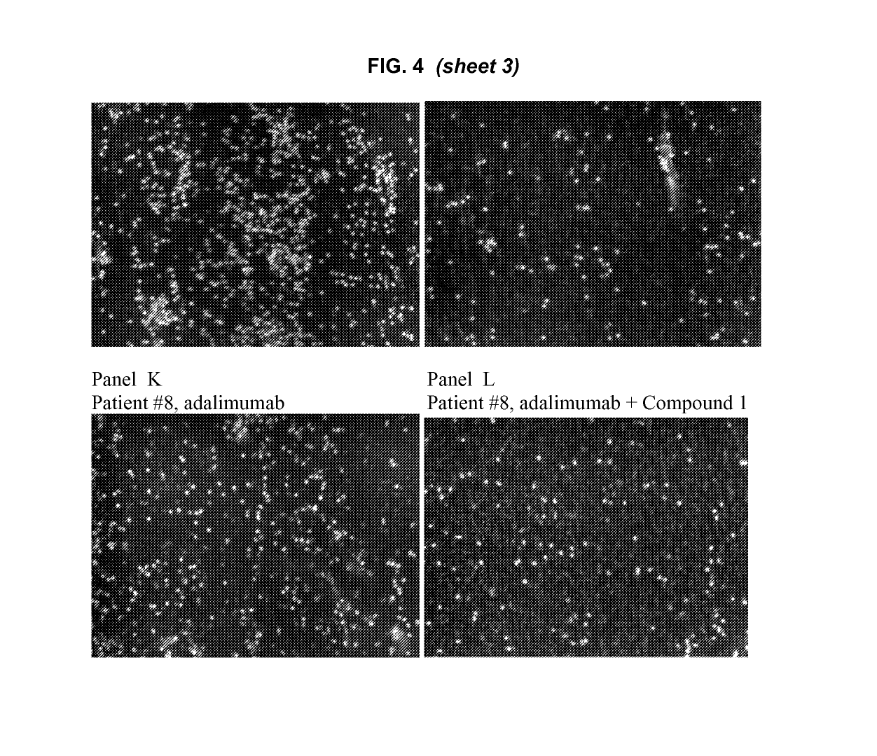

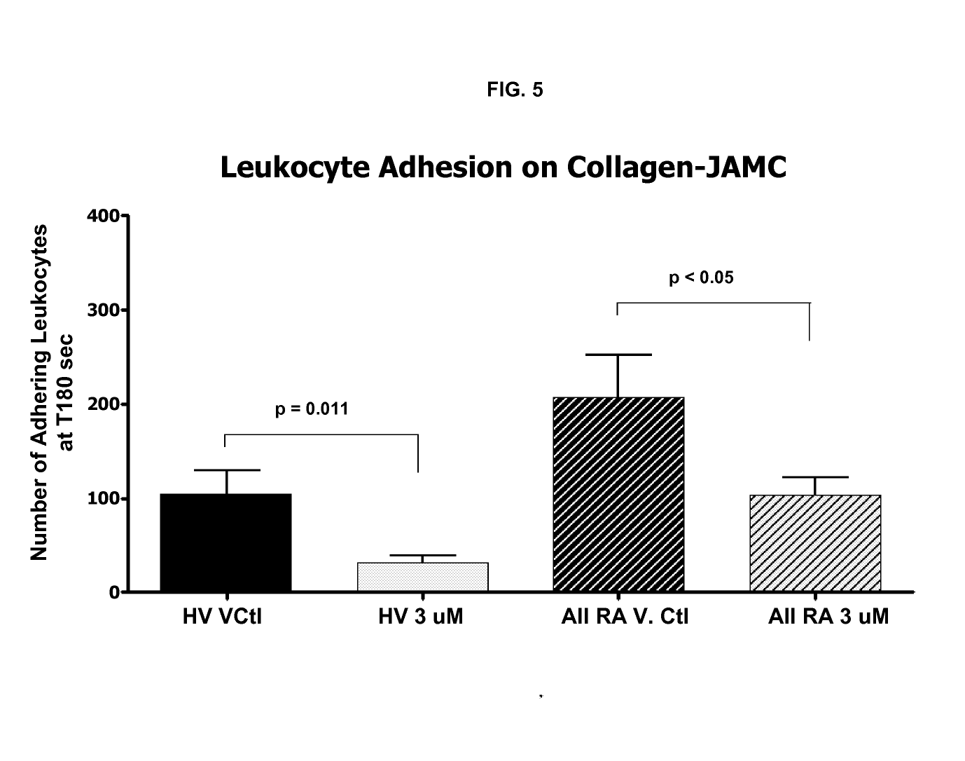

Leukocyte rolling and adhesion in human whole blood (from healthy normal volunteers or patients with rheumatoid arthritis) was monitored using real time fluorescence microscopy. Citrate anticoagulated whole blood, containing magnesium chloride, anti-platelet agent integrilin, vehicle control or Compound 1, was perfused over a mixed collagen type 1+JAM-C surface at a shear rate of 300/sec. Prior to experimentation, leukocytes were labeled in whole blood with Rhodamine 6G to permit visualization by fluorescence microscopy. The increase in fluorescence intensity is proportional to the amount of fluorescently labeled leukocytes recruited under the area of observation. Baseline and samples with Compound 1 (3 uM) are presented in single image snapshots at 180 sec and 360 sec across the treatment groups. For quantitation of inhibitory activity (TABLE), data are presented as percentage increase in fluorescence intensity over baseline 180 and 360 sec post initiation of the blood perfusion (see FIG. 4).

| | Control | Compound | Control | Compound | |
|---|---|---|---|---|---|
| Patient | Vehicle | 1 at | vehicle | 1 at | Concomitant |
| ID | at 180 sec | 180 sec | At 360 sec | 360 sec | Medication |
| #10 | 44% | 29% | 107% | 86% | Methotrexate |
| #11 | 165% | 69% | 178% | 183% | Methotrexate + |
| | | | | | prednisone |
| #12 | 61% | 17% | 131% | 53% | Etanercept |
| #8 | 51% | 7% | 94% | 63% | Adalimumab |

As shown in Panels A and B, addition of Compound 1 reduces the number of leukocytes adhering to the collagen and JAM-C coated surface in whole blood from healthy donors without inflammatory disease. The rest of the panels depict profiles in RA patients which show residual leukocyte mediated activity even when the patient is treated with MTX, steroid, NSAID or anti TNF biologics. In vitro addition of Compound 1 showed inhibition of this proinflammatory activity.

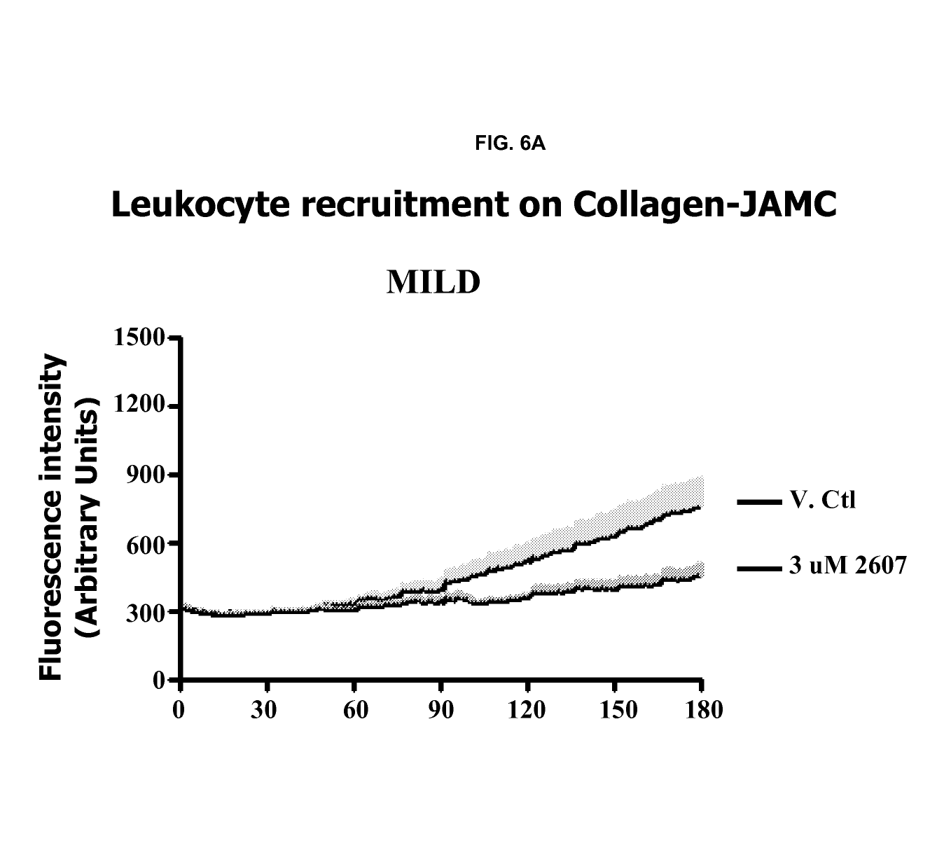

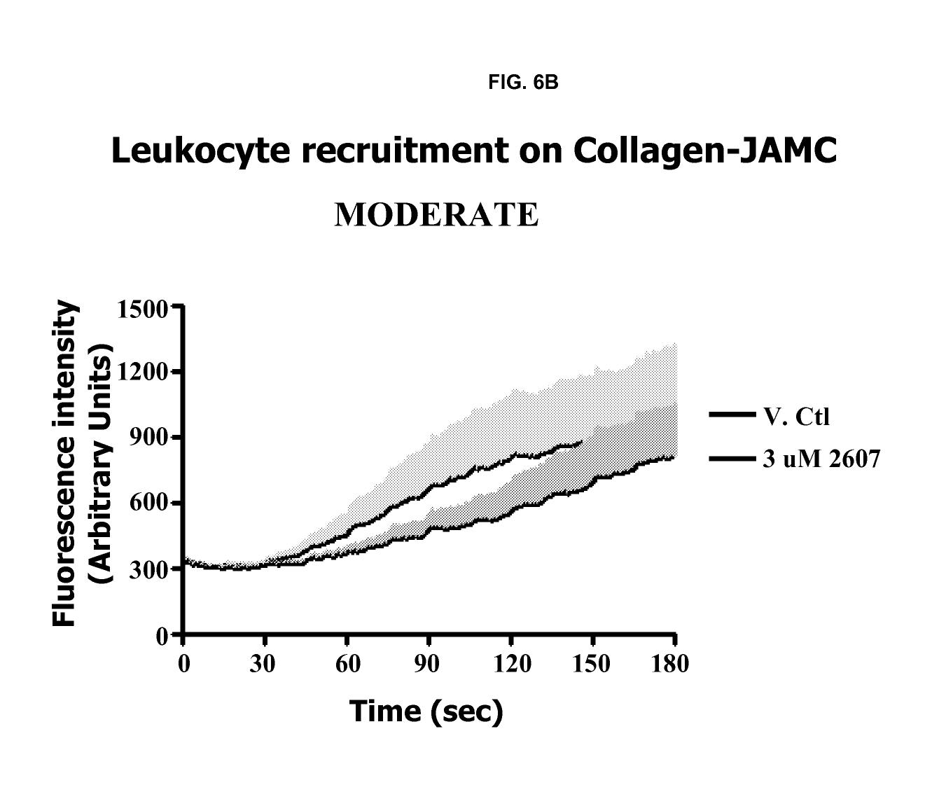

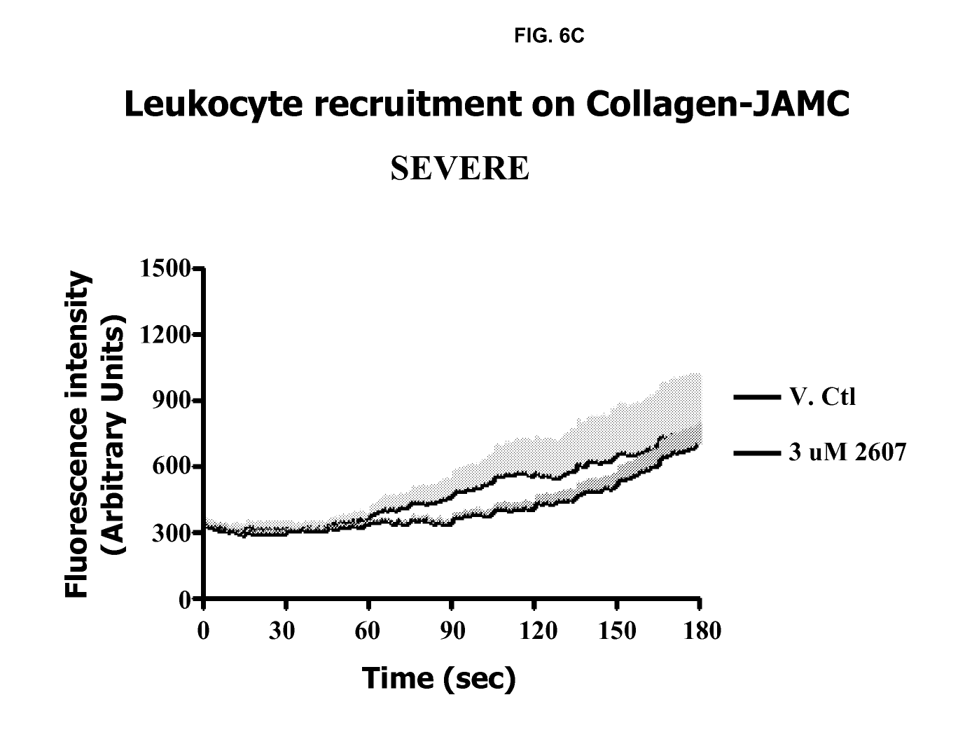

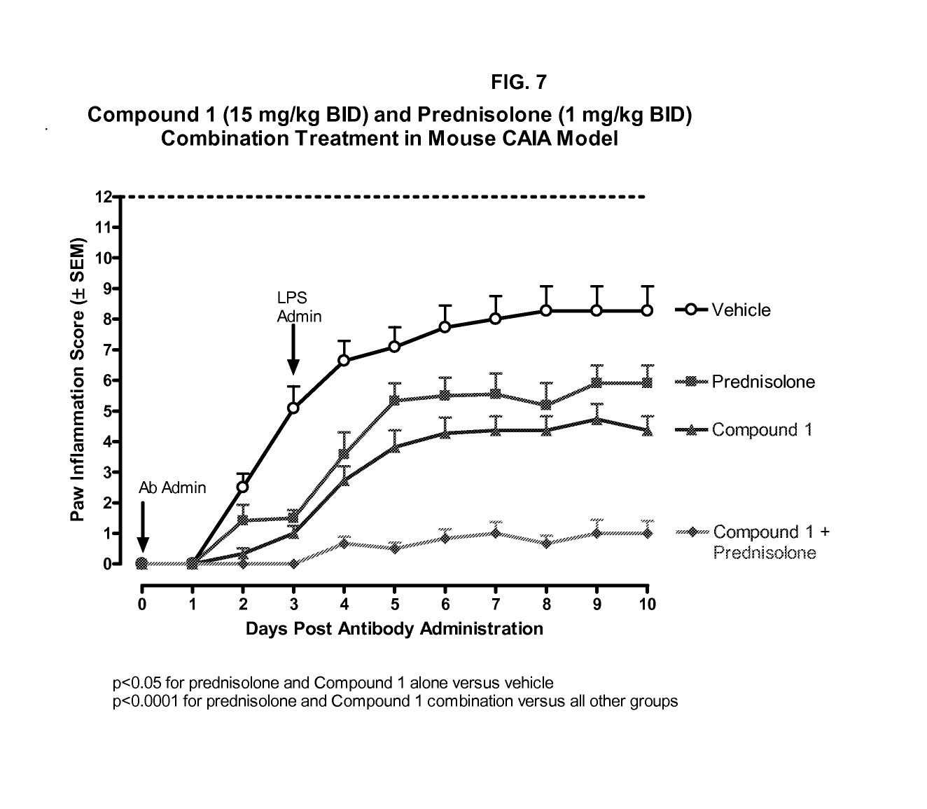

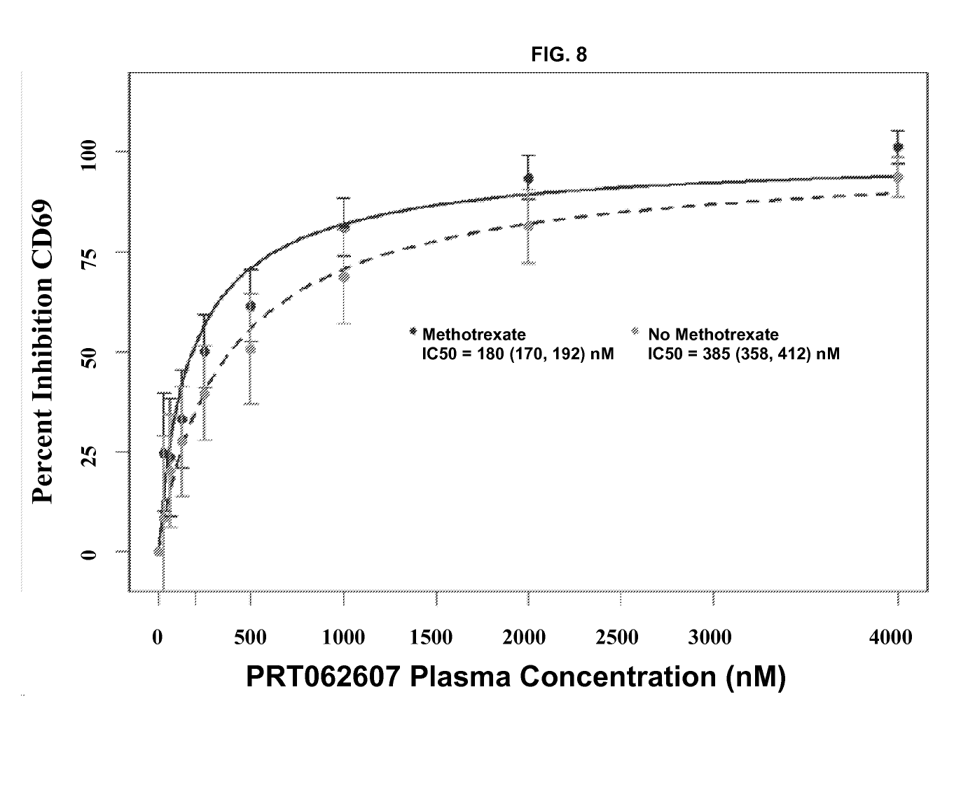

In addition, FIG. 6 shows that the addition of Compound 1 has the additional benefit of reducing leukocyte adhesion and migration in collagen and JAM-C coated surface. This effect was in all RA patient blood studied (mild, moderate and severe RA)

The present invention provides a number of embodiments. It is apparent that the examples may be altered to provide other embodiments of this invention. Therefore, it will be appreciated that the scope of this invention is to be defined by the appended claims rather than by the specific embodiments, which have been represented by way of example.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety. From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A composition for treating a cell proliferative disorder selected from the group consisting of leukemia, a lymphoma, myeloproliferative disorders, hematological malignancies, and chronic idiopathic myelofibrosis comprising administering to said mammal a therapeutically effective amount of an agent 4-(3-(2H-1,2,3-triazol-2-yl)phenylamino)-2-((1R,2S)-2-aminocyclohexylamino)pyrimidine-5-carboxamide, or a pharmaceutically acceptable salt thereof; and an antineoplastic agent.

2. The composition of claim 1, wherein the antineoplastic agent is selected from the group consisting of a topoisomerase II inhibitor, aribonucleotide reductase inhibitor, a DNA polymerase inhibitor, and combinations thereof.

3. The composition of claim 1, wherein the antineoplastic agent is selected from the group consisting of azacitidine, cladribine, decitabine. gemcitabine, mercaptopurine, thioguanine, clofarabine, troxacitabine, and pentostatin, Methotrexate (MTX), sulfosalazine, Dexamethasone, Bendamustine, Chlorambucil, gemcitabin, Doxorubicine, prednisone, vincristine, Anti CD20 antibody (other than rituximab/rituxan), Anti CD52 antibody (alemtuzumab), and Ofatumumab.

4. The composition claim 1, wherein the antineoplastic agent is selected from Doxorubicin, Idarubicine and ara-C, or a pharmaceutically acceptable salt thereof.

5. The composition claim 1, wherein the pharmaceutically acceptable salt of 4-(3-(2H-1,2,3-triazol-2-yl)phenylamino)-2-((1R,2S)-2-aminocyclohexylamino)pyrimidine-5-carboxamide is the hydrochloride or acetate salt.

6. The composition of claim 1, further comprising administering a therapeutic agent selected from the group consisting of: an anti-inflammatory agent, an immunosuppressant agent, an antimetabolic agent a hormone; an antimicrobial agent; another antineoplastic agent and combinations thereof.

7. The composition of claim 1, wherein the therapeutic agent is selected from the group consisting of: cyclophosphamide, mitoxantrone, dexamethasone, rituximab, cytarabine, granulocyte colony-stimulating factor; co-trimoxazole, and pentamidine.

8. The composition of claim 1, wherein at least one of the agents is in a sub-therapeutic dosage.

9. The composition of claim 1, wherein at least two of the agents are in sub-therapeutic dosages.

10. (canceled)

11. (canceled)

12. (canceled)

13. (canceled)

14. (canceled)

15. A method for treating a cell proliferative disorder selected from the group consisting of leukemia, a lymphoma, myeloproliferative disorders, hematological malignancies, and chronic idiopathic myelofibrosis comprising administering to a mammal a therapeutically effective amount of an agent 4-(3-(2H-1,2,3-triazol-2-yl)phenylamino)-2-((1R,2S)-2-aminocyclohexylamino)pyrimidine-5-carboxamide, or a pharmaceutically acceptable salt thereof; and an antineoplastic agent.

16. The method of claim 15, wherein the antineoplastic agent is selected from the group consisting of a topoisomerase II inhibitor, aribonucleotide reductase inhibitor, a DNA polymerase inhibitor, and combinations thereof.

17. The method of claim 15, wherein the antineoplastic agent is selected from the group consisting of azacitidine, cladribine, decitabine, gemcitabine, mercaptopurine, thioguanine, clofarabine, troxacitabine, and pentostatin, Methotrexate (MTX), sulfosalazine, Dexamethasone, Bendamustine, Chlorambucil, gemcitabin, Doxorubicine, prednisone, vincristine, Anti CD20 antibody (other than rituximab/rituxan), Anti CD52 antibody (alemtuzumab), and Ofatumumab.

18. The method of claim 15, wherein the antineoplastic agent is Methotrexate (MTX).

19. The method of claim 15, wherein the method is used in radiation therapy.

20. The method of claim 15, wherein the antineoplastic agent is selected from Idarubicine and ara-C, or a pharmaceutically acceptable salt thereof.

21. The method of claim 15, wherein the pharmaceutically acceptable salt of 4-(3-(2H-1,2,3-triazol-2-yl)phenylamino)-2-(1R,2S)-2-aminocyclohexylamino)pyrimidine-5-carboxamide is the hydrochloride or acetate salt.

22. The method of claim 15, further comprising administering a therapeutic agent selected from the group consisting of: an anti-inflammatory agent, an immunosuppressant agent, an antimetabolic agent a hormone; an antimicrobial agent; another antineoplastic agent and combinations thereof.

23. The method of claim 22, wherein the therapeutic agent is an anti-inflammatory agent selected from the group consisting of tofacitinib (CP-690550), tasocitinib, VX-509, Ruxolitinib (INCB18424), fostamatinib (R788) and combinations thereof.

24. The method of claim 22 wherein the therapeutic agent is selected from the group consisting of: doxorubicin, cyclophosphamide, mitoxantrone, dexamethasone, rituximab, cytarabine, granulocyte colony-stimulating factor; co-trimoxazole, and pentamidine.

25. (canceled)

26. (canceled)

27. (canceled)

28. (canceled)

29. (canceled)

30. (canceled)

31. (canceled)

32. (canceled)

33. (canceled)

34. A kit comprising a composition of claim 1.

35. (canceled)

36. (canceled)

37. (canceled)
